SEQUENCE LISTING (2 sequences)

SEQ ID NO: 1 - DNA, 20 bp, synthetic construct
  misc_feature 1..20: note = P5 primer
  source 1..20: mol_type = other DNA
aatgatacggcgaccaccga

SEQ ID NO: 2 - DNA, 21 bp, synthetic construct
  misc_feature 1..21: note = P7 Primer
  source 1..21: mol_type = other DNA
caagcagaagacggcatacga

(19) United States
(12) Patent Application Publication
(10) Pub. No.: US 2023/0287476 A1
(43) Pub. Date: Sep. 14, 2023

(54) Beads as Transposome Carriers

(71) Applicant: Illumina, Inc., San Diego, CA (US)

(72) Inventors: Yir-Shyuan Wu, Albany, CA (US); Tarun Kumar Khurana, Fremont, CA (US); Elisabet Rosàs-Canyelles, El Cerrito, CA (US); Fei Shen, San Diego, CA (US); Jeffrey Brodin, San Diego, CA (US); Lena Storms, San Diego, CA (US); Jeffrey S. Fisher, San Diego, CA (US)

(73) Assignee: Illumina, Inc., San Diego, CA (US)

(21) Appl. No.: 18/151,128

(22) Filed: Jan. 6, 2023

Related U.S. Application Data
(63) Continuation of application No. PCT/US2021/040612, filed on Jul. 7, 2021.
(60) Provisional application No. 63/049,172, filed on Jul. 8, 2020.

(51) Int. Cl. C12Q 1/6806; B82Y 5/00; C12Q 1/6834

(52) U.S. Cl. CPC C12Q 1/6806; B82Y 5/00; C12Q 1/6834

(57) ABSTRACT

Degradable polyester bead are described comprising a plurality of transposome complexes immobilized to the surface thereof, wherein each transposome complex comprises a transposase bound to a first polynucleotide and a second polynucleotide, wherein the first polynucleotide comprises a 3′ portion comprising a transposon end sequence and a tag, and the second polynucleotide comprises a 5′ portion that is complementary to and hybridized to the transposon end sequence, and wherein the polyester bead has a melting point of from 50° C. to 65° C. Flow cells and methods related to these polyester beads are described. Also described herein are compositions comprising a bead and at least one nanoparticle and methods of use of such compositions comprising transposome complexes immobilized to nanoparticles.

### CROSS-REFERENCE TO RELATED APPLICATIONS

This application is a bypass continuation of PCT/US2021/040612, filed Jul. 7, 2021, which claims the benefit of priority of U.S. Provisional Application No. 63/049,172, filed Jul. 8, 2020, the contents of which are each incorporated by reference herein in their entireties for any purpose.

### SEQUENCE LISTING

The present application is filed with a Sequence Listing in electronic XML format. The Sequence Listing is provided as a file entitled “2023-04-06_01243-0018-00US_ST26” created on Apr. 6, 2023, which is 3,597 bytes in size. The information in the electronic format of the sequence listing is incorporated herein by reference in its entirety.

### DESCRIPTION

### Field

This application relates to degradable polyester beads as transposome carriers and to flow cells comprising these beads. These beads may be used in a variety of methods to prepare sequencing libraries.

### Background

Bead-linked transposomes are used in a variety of methods to prepare libraries for sequencing. In some systems, non-degradable M-280 magnetic beads (Dynabeads®, Thermo Fisher) are used for library clean-up as solid-phase reversible immobilization (SPRI) beads or used as transposomes carriers to allow on-bead library preparation for long DNA molecules and to control delivery of the produced DNA library directly into flow cells. However, M-280 beads may become entrained in downstream tubing and valves of the sequencers and cause clogging or damage of instruments during automated preparations.

Degradable hydrogel beads have been described that can encapsulate genetic material and allow capture on a surface of a sequencing flow cell. However, these hydrogel beads surround or encapsulate genetic material and are then degraded in the presence of a liquid diffusion barrier or immiscible fluid (See PCT Application Nos. PCT/US18/44646 and PCT/US18/44855). Hydrogel beads may be porous and allow permeabilization of enzymes beyond the bead surface. Alternative types of beads are needed to support a variety of different sequencing formats, such as beads that allow coupling on the bead surface (such as surface coupling of transposome complexes and/or target nucleic acid for tagmentation reactions).

Described herein are degradable polycaprolactone (PLC) beads for use as transposome carriers to improve methods that include automated preparations. For example, streptavidin can be conjugated to the surface of PLC microsphere surfaces and allow assembly of biotin-conjugated transposomes on PLC microsphere surfaces and also allow attachment of PLC beads on biotinylated flow cell surface for localized library release and clustering. After library release and seeding to flow cell surface, the PLC beads can be selectively degraded to avoid any potential damage or clogging of a sequencer fluidic system. In this way, methods incorporating the present beads allow tagmentation on the bead surface and subsequent sequencing library release in close proximity to a flow cell, without having a negative impact on downstream processes.

Also disclosed herein are methods of library preparation using compositions comprising a bead and at least one nanoparticle. Library preparation is an important initial step for all next-generation sequencing (NGS) applications. Bead-linked transposome (BLT) library construction greatly improved the DNA library preparation process by eliminating the need for a separate DNA fragmentation step and removing the prerequisite for ligation between DNA fragments. However, the insert size of the library of BLT depends on the distribution of the Tn5 transposome on the beads. Single- or double-sided size selection (such as with SPRI beads) is required to remove the too short (low output, short reads) or too long (poorly clustering) libraries. The concentration and size of these size selected library samples then need to be quantified after size selection, and the libraries need to be diluted or normalized to appropriate concentration following by denaturation. A library is then seeded on a flow cell before the amplification reaction (clustering). Significant portion of library samples are consumed during all these steps, while only a relatively small percentage of library samples can be sequenced. As described herein, compositions comprising a bead and at least one nanoparticle can be used in improved methods of library generation.

### SUMMARY

In accordance with the description, described herein are degradable polyester beads. Also described herein are compositions comprising a bead and at least one nanoparticle.

Embodiment 1. A degradable polyester bead comprising a plurality of transposome complexes immobilized to the surface thereof, wherein each transposome complex comprises a transposase bound to a first polynucleotide and a second polynucleotide, wherein the first polynucleotide comprises a 3′ portion comprising a transposon end sequence and a tag, and the second polynucleotide comprises a 5′ portion that is complementary to and hybridized to the transposon end sequence, and wherein the polyester bead has a melting point of from 50° C. to 65° C., or 60° C.

Embodiment 2. The degradable polyester bead of embodiment 1, wherein the polyester bead comprises polycaprolactone.

Embodiment 3. The degradable polyester bead of embodiment 1 or embodiment 2, comprising a plurality of magnetic nanoparticles immobilized thereto, optionally wherein the magnetic nanoparticles are beads with a magnetic core, optionally wherein the magnetic core comprises iron, nickel, and/or cobalt.

Embodiment 4. The polyester bead of any one of embodiments 1 to 3, wherein each transposome complex comprises a polynucleotide binding moiety, the bead comprises a plurality of bead binding moieties covalently bound to the surface thereof, and the transposome complexes are immobilized to the bead surface through binding of the polynucleotide binding moieties to the bead binding moieties.

Embodiment 5. The polyester bead of embodiment 4, wherein each polynucleotide binding moiety is covalently bound to the first polynucleotide of each transposome complex.

Embodiment 6. The polyester bead of embodiment 4, wherein each polynucleotide binding moiety is covalently bound to the second polynucleotide of each transposome complex.

Embodiment 7. The polyester bead of any one of embodiments 4 to 6, wherein the bead binding moiety is streptavidin or avidin and the polynucleotide binding moiety is biotin.

Embodiment 8. The polyester bead of any one of embodiments 4 to 7, wherein each bead binding moiety is covalently bound to the polyester bead through a linker, wherein the linker optionally comprises —N═CH—(CH2)3-CH═N—, —C(O)NH—(CH2)6-N═, or —C(O)NH—(CH2)6-N═CH—(CH2)3CH═N—.

Embodiment 9. The polyester bead of any one of embodiments 3 to 6, wherein each magnetic nanoparticle is covalently bound to the polyester bead through a linker, wherein the linker optionally comprises —N═CH—(CH2)3-CH═N—, —C(O)NH—(CH2)6-N═, or —C(O)NH—(CH2)6-N═CH—(CH2)3CH═N—.

Embodiment 10. The polyester bead of any one of the preceding embodiments, wherein the polyester bead is immobilized on the surface of a flow cell.

Embodiment 11. The polyester bead of embodiment 10, wherein the polyester bead is immobilized on the surface of the flow cell through covalent binding of a bead binding moiety to a flow cell binding moiety on the surface of the flow cell.

Embodiment 12. The polyester bead of embodiment 11, wherein the polynucleotide binding moiety and the flow cell binding moiety are the same type of binding moiety, and the transposome complexes are bound to a first portion of the bead binding moieties on the bead and the flow cell binding moiety is bound to a second portion of the bead binding moieties on the bead.

Embodiment 13. The polyester bead of any of the preceding embodiments, comprising a target nucleic acid or one or more fragments thereof, optionally wherein most transposome complexes are immobilized on the surface of the bead.

Embodiment 14. A flow cell comprising a polyester bead immobilized to the surface of the flow cell, wherein the polyester bead comprises a plurality of transposome complexes immobilized to the surface thereof, wherein each transposome complex comprises a transposase bound to a first polynucleotide and a second polynucleotide, wherein the first polynucleotide comprises a 3′ portion comprising a transposon end sequence and a tag, and the second polynucleotide comprises a 5′ portion that is complementary to and hybridized to the transposon end sequence; and wherein the polyester bead has a melting point of from 50° C. to 65° C., or 60° C.

Embodiment 15. The flow cell of embodiment 14, wherein the polyester bead comprises polycaprolactone.

Embodiment 16. The flow cell of embodiment 14 or embodiment 15, wherein the polyester bead comprises a plurality of immobilized nanoparticles immobilized thereto.

Embodiment 17. The flow cell of any one of embodiments 14 to 16, wherein each transposome complex comprises a polynucleotide binding moiety, the bead comprises a plurality of bead binding moieties covalently bound to the surface thereof, and the transposome complexes are immobilized to the bead surface through binding of the polynucleotide binding moieties to the bead binding moieties.

Embodiment 18. The flow cell of embodiment 17, wherein each polynucleotide binding moiety is covalently bound to the first polynucleotide of each transposome complex.

Embodiment 19. The flow cell of embodiment 17, wherein each polynucleotide binding moiety is covalently bound to the second polynucleotide of each transposome complex.

Embodiment 20. The flow cell of any one of embodiments 17 to 19, wherein the bead binding moiety is streptavidin or avidin and the polynucleotide binding moiety is biotin.

Embodiment 21. The flow cell of any one of embodiments 17 to 20, wherein each bead binding moiety is covalently bound to the polyester bead through a linker, wherein the linker optionally comprises —N═CH—(CH2)3-CH═N—, —C(O)NH—(CH2)6-N═, or —C(O)NH—(CH2)6-N═CH—(CH2)3CH═N—.

Embodiment 22. The flow cell of any one of embodiments 16 to 21, wherein each magnetic nanoparticle is covalently bound to the polyester bead through a linker, wherein the linker optionally comprises —N═CH—(CH2)3-CH═N—, —C(O)NH—(CH2)6-N═, or —C(O)NH—(CH2)6-N═CH—(CH2)3CH═N—.

Embodiment 23. The flow cell of any one of embodiments 14 to 22, wherein the polyester bead is immobilized on the surface of the flow cell through covalent binding of a bead binding moiety to a flow cell binding moiety on the surface of the flow cell, or the bead comprises a plurality of immobilized magnetic nanoparticles immobilized thereto, optionally wherein the magnetic nanoparticles are used for seeding the polyester bead to a surface of the flow cell.

Embodiment 24. The flow cell of embodiment 23, wherein the polynucleotide binding moiety and the flow cell binding moiety are the same type of binding moiety, and the transposome complexes are bound to a first portion of the bead binding moieties on the bead and the flow cell binding moiety is bound to a second portion of the bead binding moieties on the bead.

Embodiment 25. The flow cell of any one of embodiments 14 to 24, comprising a target nucleic acid or one or more fragments thereof, optionally wherein most transposome complexes are immobilized on the surface of the bead.

Embodiment 26. A method of preparing a nucleic acid library from a target nucleic acid comprising contacting the target nucleic acid with the polyester bead of any one of embodiments 1-12 or the flow cell of any one embodiments 14-25 under conditions whereby the target nucleic acid is fragmented by the transposome complexes and the 3′ transposon end sequence of the first polynucleotide is transferred to a 5′ end of at least one strand of the fragments, thereby producing an immobilized library of fragments wherein at least one strand is 5′-tagged with the tag.

Embodiment 27. The method of embodiment 26, wherein contacting comprises contacting the target nucleic acid with the polyester bead of any one of embodiments 1 to 8, and the method comprises immobilizing the bead comprising the immobilized library of fragments to the surface of a flow cell.

Embodiment 28. The method of embodiment 26 or embodiment 27, wherein the bead is immobilized to the surface of the flow cell through binding of the bead binding moiety to a flow cell binding moiety on the surface of the flow cell, or the bead comprises a plurality of immobilized magnetic nanoparticles immobilized thereto, optionally wherein the magnetic nanoparticles are used for seeding the polyester bead to a surface of the flow cell.

Embodiment 29. The method of embodiment 22, wherein contacting comprises contacting the target nucleic acid with the polyester bead of any one of embodiments 10 to 12.

Embodiment 30. The method of any one of embodiments 27 to 29, comprising releasing the fragments from the immobilized bead to provide a spent bead and capturing the released fragments on the flow cell surface to produce captured fragments.

Embodiment 31. The method of embodiment 30, wherein releasing the fragments from the immobilized bead comprises amplifying the fragments off the bead.

Embodiment 32. The method of embodiment 30 or embodiment 31, wherein capturing the released fragments comprises hybridizing the released fragments to capture oligonucleotides on the surface of the flow cell.

Embodiment 33. The method of embodiment any one of embodiments 30 to 32, comprising amplifying the captured fragments on the flow cell surface to produce immobilized, amplified fragments.

Embodiment 34. The method of embodiment 33, wherein amplifying the captured fragments comprises bridge amplification to produce clusters of the fragments.

Embodiment 35. The method of any one of embodiments 30 to 34, comprising detaching the spent bead from the flow cell surface by treating the spent bead with an excess of solution-phase flow cell binding moiety to provide a solution-phase spent bead.

Embodiment 36. The method of embodiment 35, comprising degrading the solution-phase spent bead with a degrading agent.

Embodiment 37. The method of embodiment 36, comprising removing the degraded bead from the flow cell.

Embodiment 38. The method of embodiment 36 or embodiment 37, wherein the degrading agent is (a) a temperature of from 50° C. to 65° C., or 60° C., and/or (b) an aqueous base.

Embodiment 39. The method of embodiment 38, wherein the aqueous base is NaOH.

Embodiment 40. The method of embodiment 39, wherein the aqueous base is 1M-5M NaOH.

Embodiment 41. The method of embodiment 40, wherein the aqueous base is 1M, 2M, 3M, 4M, or 5M NaOH.

Embodiment 42. The method of embodiment 40, wherein the aqueous base is 3M NaOH.

Embodiment 43. The method of any one of embodiments 33 to 42, comprising sequencing the immobilized, amplified fragments or the clusters of the fragments.

Embodiment 44. A method of making a polyester bead of any one of embodiments 1 to 9 comprising immobilizing a plurality of transposome complexes to a polyester bead, wherein each transposome complex comprises a transposase bound to a first polynucleotide and a second polynucleotide, wherein the first polynucleotide comprises a 3′ portion comprising a transposon end sequence and a tag, and the second polynucleotide comprises a 5′ portion that is complementary to and hybridized to the transposon end sequence.

Embodiment 45. The method of embodiment 44, comprising immobilizing a plurality of magnetic nanoparticles to the polyester bead.

Embodiment 46. A composition comprising a bead and a nanoparticle, wherein the bead comprises a functional group that is capable of binding to the nanoparticle, optionally wherein the nanoparticle or the bead is magnetic.

Embodiment 47. The composition of embodiment 46, wherein the nanoparticle is a synthetic dendron, a DNA dendron, or a polymer brush; and/or the nanoparticle comprises a hardcore bead, optionally wherein the nanoparticle has a diameter of 50-150 nm, further optionally wherein the nanoparticle has a diameter of 100 nm.

Embodiment 48. The composition of any one of embodiments 1-47, wherein the nanoparticle comprises a single immobilized transposome complex, or more than one immobilized transposome complex, optionally wherein the more than one immobilized transposome complexes are immobilized with similar distances between each transposome complex on the nanoparticle.

Embodiment 49. The composition of embodiment 48, wherein the immobilized transposome complex or transposome complexes are oriented with the transposase facing away from the nanoparticle.

Embodiment 50. The composition of embodiment 48 or embodiment 49, wherein the transposome complex is immobilized to the nanoparticle by (a) binding of a transposon comprising biotin, desthiobiotin, or dual biotin to avidin or streptavidin comprised on the nanoparticle, or (b) a click chemistry reaction between an agent comprised in a transposon and an agent comprised in the nanoparticle, optionally wherein the click chemistry reaction is a reaction between an azide on the nanoparticle and a dibenzylcyclooctyne (DBCO) on the transposon.

Embodiment 51. The composition of embodiment any one of embodiments 46-49, wherein the bead is a carrier bead that can bind multiple nanoparticles, optionally wherein the bead has a diameter of 1 μm or larger and/or the bead is a degradable polyester bead of any one of embodiments 1-8.

Embodiment 52. The composition of any one of embodiments 46-50, wherein the functional group is a chemical attachment handle and/or a clustering primer, optionally wherein (a) the chemical attachment handle and/or clustering primer directly binds to the nanoparticle; (b) the chemical attachment handle and/or clustering primer indirectly binds to the nanoparticle; or (c) a chemically modified oligonucleotide binds to both the clustering primer comprised in the bead and to the nanoparticle.

Embodiment 53. The composition of any one of embodiments 46-52, wherein the interaction between the nanoparticle and the bead is a reversible and/or non-covalent interaction, optionally wherein the reversible and/or non-covalent interaction is a protein-ligand interaction or a metal-chelator interaction, further optionally wherein the protein-ligand interaction is a biotin-streptavidin interaction or the metal-chelator interaction is nickel-polyhistidine or cobalt-polyhistidine interaction.

Embodiment 54. The composition of any one of embodiments 46-53, wherein the bead comprises a clustering primer and the nanoparticle comprises an immobilized oligonucleotide, optionally wherein the immobilized oligonucleotide and the clustering primer bind directly to each other or a linking oligonucleotide is capable of binding to both the immobilized oligonucleotide and the clustering primer.

Embodiment 55. The composition of any one of embodiments 46-52, wherein the interaction between the nanoparticle and the bead is an irreversible and/or covalent interaction, optionally wherein the covalent interaction is a cleavable linker between the bead and the nanoparticle, further optionally wherein the cleavable linker is a chemically or enzymatically cleavable linker.

Embodiment 56. A method of seeding a flow cell comprising (a) dissociating the bead and the nanoparticle of the composition of any one of embodiments 46-55, optionally wherein dissociating the bead and the nanoparticle is by cleavage of a cleavable linker or by dissociation of a reversible and/or non-covalent interaction between the nanoparticle and the bead; and (b) immobilizing the nanoparticle on the surface of a flow cell.

Embodiment 57. A flow cell comprising a nanoparticle immobilized to its surface prepared by the method of embodiment 56 or a flow cell comprising a composition of any one of embodiments 46-55 immobilized to the surface of the flow cell, optionally wherein the composition is immobilized to the flow cell through binding of the nanoparticle to the surface of the flow cell.

Embodiment 58. The flow cell of embodiment 57, comprising a target nucleic acid or one or more fragments thereof, each bound to at least two transposome complexes immobilized on nanoparticles.

Embodiment 59. A method of preparing a nucleic acid library from a target nucleic in a reaction solution comprising contacting the target nucleic acid with a mixture of compositions each comprising a bead and a nanoparticle of any one of embodiments 48-55 under conditions whereby the target nucleic acid is fragmented by the transposome complexes and the 3′ transposon end sequence of the first polynucleotide is transferred to a 5′ end of the fragment, thereby producing immobilized double stranded target nucleic acid fragments wherein one strand is 5′-tagged with the tag.

Embodiment 60. The method of embodiment 59, further comprising (a) adding a sodium dodecyl sulfate (SDS) solution after the producing of fragments, wherein the SDS stops the producing of additional fragments; or (b) releasing fragments from the transposome complexes after the producing of fragments or after adding the SDS solution, optionally wherein the releasing is performed at a temperature of 80° C. or by amplifying.

Embodiment 61. The method of embodiment 60, wherein releasing the fragment from the transposome complexes releases the fragments from the nanoparticle, optionally wherein the fragments are in solution after the releasing.

Embodiment 62. The method of embodiment 61, further comprising removing beads from the reaction solution after the releasing of fragments, optionally wherein (a) the beads are magnetic and the removing beads is performed using a magnetic field, or (b) the beads are degradable polyester beads and the removing beads is performed using a degrading agent, optionally wherein the degrading agent is (i) a temperature of from 50° C. to 65° C., or 60° C., and/or (ii) an aqueous base.

Embodiment 63. The method of any one of embodiments 59-62, wherein (a) fragments in solution are amplified, and the amplified fragments are loaded into a flow cell, captured, and sequenced; or (b) fragments immobilized to a mixture of compositions comprising beads and nanoparticles are loaded into a flow cell, and fragments are released and/or beads are removed, and fragments are captured on the flow cell, amplified, and sequenced, optionally wherein fragments released from a single composition will be captured in spatial proximity on the flow cell.

Embodiment 64. The method of any one of embodiments 59-63, wherein a target nucleic acid is fragmented by multiple transposome complexes, optionally wherein all the transposome complexes are identical and the fragments are tagged with the same adapter sequence at the 5′ end of both strands of the double-stranded fragments.

Embodiment 65. The method of embodiment 64, further comprising (a) releasing the double-stranded target nucleic acid fragments from the transposome complexes, optionally wherein the fragments are then immobilized to a solid support, (b) hybridizing a polynucleotide comprising an adapter sequence and a sequence all or partially complementary to the first 3′ end transposon sequence, wherein the adapter sequence comprised in the polynucleotide is different from the adapter sequence comprised in the transposome complexes, (c) optionally extending a second strand of the double-stranded target nucleic acid fragments, (d) optionally ligating the polynucleotide or extended polynucleotide with the double-stranded target nucleic acid fragments, and (e) producing double-stranded fragments.

Embodiment 66. The method of embodiment 65, wherein the polynucleotide further comprises a UMI and the double-stranded target nucleic acid fragments comprise the UMI, optionally wherein the UMI is located directly adjacent to the 3′ end of the target nucleic acid fragment.

Embodiment 67. The method of embodiment 65 or embodiment 66, wherein double stranded fragments produced are tagged with a first-read sequence adapter sequence from the first transposon at the 5′ end of one strand and with a second-read sequence adapter sequence from the polynucleotide at the 5′ end of the other strand.

Embodiment 68. The method of embodiment 67, further comprising (a) releasing the double-stranded fragments from the transposome complex, optionally wherein the fragments are immobilized to a solid support, (b) hybridizing a first polynucleotide comprising an adapter sequence, wherein the adapter in the first transposon is different from the adapter in the first polynucleotide, (c) optionally adding a second polynucleotide comprising regions complementary to the first polynucleotide to produce a double-stranded adapter, (d) optionally extending a second strand of the double-stranded target nucleic acid fragments, (e) optionally ligating the double-stranded adapter with the double-stranded target nucleic acid fragments, and (f) producing double stranded fragments.

Embodiment 69. The method of embodiment 68, wherein the first polynucleotide further comprises a UMI and the double-stranded fragments comprise the UMI, optionally wherein the UMI is located between the target nucleic acid fragment and the adapter sequence from the first polynucleotide.

Embodiment 70. The method of embodiment 68 or embodiment 69, wherein double stranded fragments produced are tagged with a first-read sequence adapter sequence from the first transposon at the 5′ end of one strand and with a second-read sequence adapter sequence from the first polynucleotide at the 5′ end of the other strand.

Embodiment 71. The method of any one of embodiments 59-70, wherein the average number of nanoparticles immobilized to a bead in the mixture of compositions comprising a bead and a nanoparticle determines the size of target nucleic acid fragments, optionally wherein the method does not require size selection of produced fragments before amplifying or sequencing.

Embodiment 72. The method of any one of embodiments 59-71, wherein steric hindrance between nanoparticles comprised on the same bead reduces generation of fragments of less than 35 base pairs, optionally wherein produced fragments are sequenced using long read sequencing.

Embodiment 73. A method of preparing a mixture of compositions comprising a bead and a nanoparticle comprising (a) mixing beads and nanoparticles to prepare compositions comprising beads and nanoparticles of any one of embodiments 46-55, optionally wherein the beads are magnetic and the mixture is performed using a magnetic field; (b) separating the beads from the mixture, optionally wherein the beads are magnetic and the separating beads is performed using a magnetic field; (c) evaluating the average number of nanoparticles associated with each bead, optionally wherein the evaluating is performed by preparing fragments according to the method of any one of embodiments 59-72 and determining fragment size; and (d) repeating prior steps until a desired average number of nanoparticles is associated with each bead in the mixture of compositions.

Additional objects and advantages will be set forth in part in the description which follows, and in part will be obvious from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate one (several) embodiment(s) and together with the description, serve to explain the principles described herein.

### BRIEF DESCRIPTION OF THE DRAWINGS

FIGS. 1A-1B provide an overview of conjugation of streptavidin and/or magnetic nanoparticles to degradable polycaprolactone (PCL) beads. (A) Surface activation of PCL beads by aminolysis. (B) Assembly of biotin-conjugated transposomes on PCL bead surface.

FIG. 2 shows overview of library seeding/clustering, release, and melt/wash steps with degradable PCL beads. After in-flow cell library release and clustering, PCL beads are released from flow cell surface by excess free biotin and melted at a temperature above 60° C. before being washed out of the flow cell.

FIG. 3 shows a target nucleic acid immobilized to transposome complexes comprised on nanoparticles, wherein multiple nanoparticles are immobilized to a single carrier bead. Such a composition comprising a bead with multiple immobilized nanoparticles allows for multiple fragments from a given target nucleic acid to be prepared on the same carrier bead.

FIGS. 4A-4C present some representative types of nanoparticles that may be comprised in compositions, such as a synthetic dendron (A), a DNA dendron (B), or a polymer brush (C).

FIGS. 5A and 5B show some representative means of immobilizing transposome complexes to nanoparticles, such as by a biotin-avidin interaction (A) or a click chemistry reaction of azide-DBCO (B), based on association of the nanoparticle with a first transposon of the transposome complex. In both FIGS. 5A and 5B, the squiggly line between P5 and A14 (FIG. 5A) and P7 and A14 (FIG. 5B) is a spacer. In FIG. 5A, the squiggly line on the nanoparticle represents avidin or streptavidin, which binds to the 3′ biotin. In FIG. 5B, the squiggly line represents azide, which binds to the 3′ DBCO in the click chemistry reaction.

FIGS. 6A-6D show embodiments of compositions of a bead (a carrier bead) and least one nanoparticle. (A) A composition comprising a bead, comprising a chemical attachment handle and a clustering primer, and a nanoparticle. (B) A composition comprising a nanoparticle comprising an immobilized oligonucleotide and a bead comprising a clustering primer, wherein the immobilized oligonucleotide can bind to the clustering primer. (C) A composition comprising a nanoparticle comprising an immobilized oligonucleotide and a bead comprising a clustering primer, wherein a linking oligonucleotide binds to both the immobilized oligonucleotide and the clustering primer. (D) A composition comprising a nanoparticle and a bead, wherein a chemically modified oligonucleotide is bound to a clustering primer on a bead and wherein the chemical modification also can bind to a functional group on a nanoparticle.

FIG. 7 shows a method of preparing compositions comprising a bead with immobilized nanoparticles.

FIG. 8 summarizes a method of preparing a sequencing library using a mixture of compositions comprising a bead with immobilized nanoparticles.

### DESCRIPTION OF THE SEQUENCES

TABLE 1
Table 1 provides a listing of certain
sequences referenced herein.
Description of the Sequences

| Description | Sequences | SEQ ID NO |
|---|---|---|
| P5 sequence | AATGATACGGCGACCACCGA | 1 |
| P7 sequence | CAAGCAGAAGACGGCATACGA | 2 |

### DESCRIPTION OF THE EMBODIMENTS

Described herein are degradable polyester beads that can comprise a plurality of transposome complexes immobilized to the bead surface. These beads may be used as transposome carriers. As used herein, a “transposome carrier” refers to an agent to which transposomes can be immobilized, wherein the agent can also facilitate release of library products in close proximity to a flow cell. Since the beads can be degraded after seeding of the library fragments on the flow cell, the beads will not interfere with downstream processes, such as sequencing. Also described herein are compositions comprising a bead and at least one nanoparticle and methods using these compositions.

### I. Compositions Comprising a Bead and at Least One Nanoparticle

In some embodiments, a composition comprises a bead and at least one nanoparticle. In some embodiments, the bead comprises a functional group that is capable of binding to the nanoparticle. In some embodiments, the nanoparticle and/or the bead is magnetic.

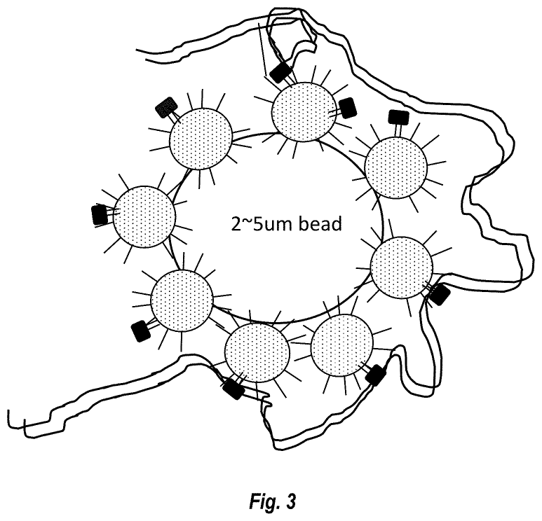

As showed in FIG. 3, instead of immobilizing Tn5 directly on the magnetic micrometer size beads in BLT, Tn5 can be immobilized on nanoparticles (“clustering nanoparticles”) and then loaded on micrometer size magnetic beads (carrier beads). The clustering nanoparticles surface are covered with a clustering oligonucleotide (for example, P7 oligonucleotide, also known as a clustering primer) for clustering and with a single Tn5 for dsDNA tagmentation. After the tagmentation, the nanoparticles can be released to the solution. Amplification (such as cluster amplification) can be performed in the solution or after the nanoparticles are loaded (i.e., captured) on a flow cell surface.

In some embodiments, compositions comprising beads and nanoparticles increase the utilization rate of sample by an integrated clustering and library preparation workflow. In some embodiments, the steric hindrance of the nanoparticles reduces the production of the short insert sizes within the library. In some embodiments, the Tn5 tagmentation could be indexed for synthetic long read sequencing applications.

A. Beads

In some embodiments, the bead comprised in a composition is a carrier bead. In some embodiments, the carrier bead binds to a number of nanoparticles.

In some embodiments, the carrier bead has a diameter of 1 μm or greater. In some embodiments, the carrier bead has a diameter of 2-5 μm. In some embodiments, the size of the bead influences how many nanoparticles are immobilized on its surface. For example, a larger bead can immobilize more nanoparticles. When the immobilized nanoparticle comprises transposome complexes (as described below), the total number of nanoparticles could dictate the total number of transposome complexes on a composition.

Any type of bead may be used as a carrier bead. In some embodiments, the carrier bead itself does not play a role in preparation of a library besides immobilizing nanoparticles on its surface. In some embodiments, the carrier bead is non-porous or mostly non-porous, thus enabling immobilization of nanoparticles on its surface. In some embodiments, the carrier bead is hollow or solid.

In some embodiments, the carrier bead is a solid bead with a magnetic core. Such magnetic beads are well-known in the art for improving mixing or purification steps, when these steps are done in the presence of a magnetic force (such as a magnetic stand or a magnetic stirrer).

In some embodiments, the carrier bead is a degradable bead. In some embodiments, the carrier bead is a degradable polyester bead as described below. Degradable beads have advantages to allow bead removal from flow cells or reaction solutions in a controlled manner.

In some embodiments, a number of clustering nanoparticles are loaded on a carrier bead, as shown in FIG. 3. As used herein, “clustering nanoparticles” refers to nanoparticles comprising a clustering primer, also known as an amplification primer. The clustering primer may also be used to facilitate binding of nanoparticles to a carrier bead. In some embodiments, multiple nanoparticles are immobilized on a bead, and each nanoparticle on the bead comprises a single transposome complex. In some embodiments, a nanoparticle may comprise more than one transposome complex. In some embodiments, fragments of a target nucleic acid are produced by multiple nanoparticles on a carrier bead, wherein the fragments are immobilized to the nanoparticles bound to the carrier bead.

1. Functional Groups

In some embodiments, the bead comprises a functional group capable of binding to a nanoparticle.

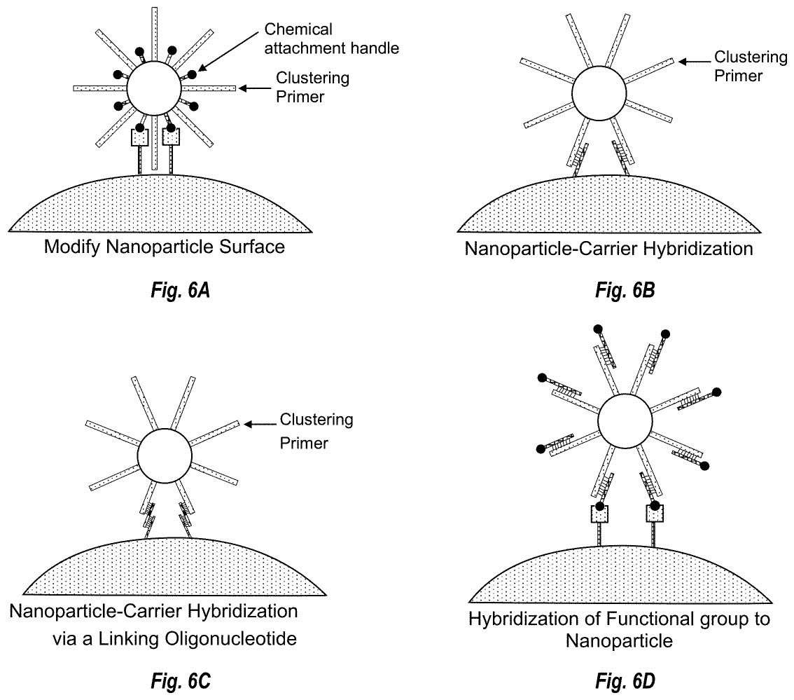

In some embodiments, the chemical attachment handle and/or clustering primer directly binds to the nanoparticle. An embodiment wherein a chemical attachment handle on a bead binds to a nanoparticle with a modified surface is shown in FIG. 6A.

In some embodiments, the clustering primer is one that can bind to nanoparticles, as well as mediate cluster amplification. In some embodiments, nanoparticles and flow cells comprise the same oligonucleotide that can bind to a clustering primer on a bead. An embodiment wherein a clustering primer on a bead binds to a nanoparticle with an immobilized oligonucleotide is shown in FIG. 6B.

In some embodiments, the chemical attachment handle and/or clustering primer indirectly binds to the nanoparticle. In some embodiments, a chemically modified oligonucleotide binds to both the clustering primer comprised in the bead and to the nanoparticle. An embodiment wherein a linking oligonucleotide binds a clustering primer on a bead and an immobilized oligonucleotide on a nanoparticle is shown in FIG. 6C.

In some embodiments a chemically modified oligonucleotide is bound to a clustering primer on a bead, wherein the chemical modification can bind to a functional group on a nanoparticle (as shown in FIG. 6D). Exemplary chemical modifications may include biotinylation, which can mediate binding to avidin or streptavidin.

In some embodiments, the interaction between the nanoparticle and the bead is a reversible and/or non-covalent interaction. In some embodiments, the reversible and/or non-covalent interaction is a protein-ligand interaction or a metal-chelator interaction. In some embodiments, the protein-ligand interaction is a biotin-streptavidin interaction or the metal-chelator interaction is nickel-polyhistidine or cobalt-polyhistidine interaction.

In some embodiments, the bead comprises a clustering primer and the nanoparticle comprises an immobilized oligonucleotide. In some embodiments, the immobilized oligonucleotide and the clustering primer bind directly to each other. In some embodiments, a linking oligonucleotide is capable of binding to both the immobilized oligonucleotide and the clustering primer, thus immobilized the nanoparticle to the bead.

In some embodiments, the interaction between the nanoparticle and the bead is an irreversible and/or covalent interaction. In some embodiments, the covalent interaction is a cleavable linker between the bead and the nanoparticle. In some embodiments, the cleavable linker is a chemically or enzymatically cleavable linker.

B. Nanoparticles

In some embodiments, nanoparticles comprised in compositions serve to immobilize an active site, such as a transposome complex, on the surface of a carrier bead. Other active sites might be envisioned, such as various enzymes. As used herein, an “active site” simply refers to a molecule that can perform a function desired by a user. In some embodiments, an active site comprises all or part of an enzyme that performs a function desired by the user.

In some embodiments, the nanoparticles have a diameter of 50 nm to 150 nm. In some embodiments, the nanoparticles have a diameter of 100 nm. In some embodiments, a nanoparticle comprises a mixture of different types of nanoparticles.

A wide variety of different nanoparticles have been described in the art. For example, nanoparticles comprising a single template site for bonding a template polynucleotide have been described in U.S. patent application Ser. No. 17/130,489 (published as US20210187469A1), Ser. No. 17/130,494 (published as US20210187470) and 62/952,799, each of which is incorporated herein by reference.

In some embodiments, the nanoparticle is a dendrimer. As used herein, a “dendrimer” refers to a nanoparticle with a branched polymeric molecule.

In some embodiments, the nanoparticle is a dendron. As used herein, a “dendron” refers to a nanoparticle that contains a single chemically addressable group, such as a focal point or core.

In some embodiments, the nanoparticle is a polymer brush. As used herein, a “polymer brush” refers to a macromolecular structure with polymer chains densely tethered to another polymer chain.

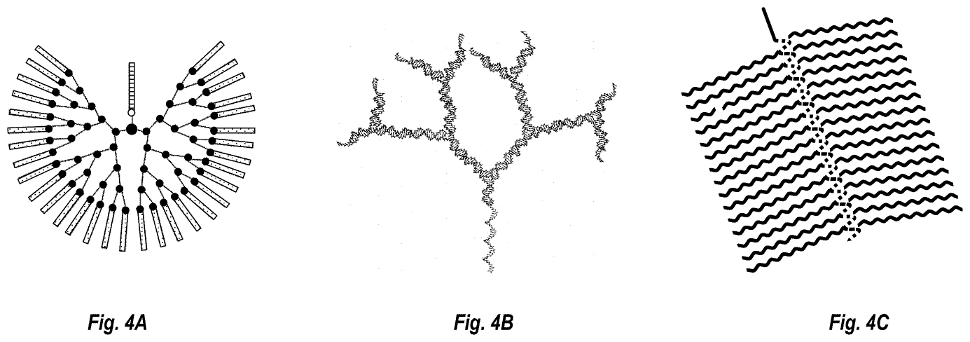

In some embodiments, the nanoparticle comprises a synthetic dendron (FIG. 4A). In some embodiments, the nanoparticle comprises a DNA dendron (FIG. 4B). In some embodiments, the nanoparticle comprises a polymer brush (FIG. 4C). One skilled in the art would be aware of a wide variety of different nanoparticles, and compositions are not limited to a specific type of nanoparticle.

In some embodiments, the nanoparticles are beads. In some embodiments, a nanoparticle is a bead that is smaller than the bead comprised in a carrier bead. In some embodiments, the nanoparticle is a hardcore bead. In some embodiments, the bead has a diameter of 20 nm-200 nm. In some embodiments, the bead has a diameter of 50 nm-150 nm. In some embodiments, the bead has a diameter of 90 nm-110 nm. In some embodiments, the bead has a diameter of 100 nm.

In some embodiments, the nanoparticles are magnetic. In some embodiments, the nanoparticles are a bead with a magnetic material core. In some embodiments, the magnetic material is iron, nickel, or cobalt. In some embodiments, the magnetic material core is coated with a silica shell. In some embodiments, the silica shell allows for functionalization with organo-silane molecules. In some embodiments, this functionalization allows for binding to functional groups comprised on carrier beads.

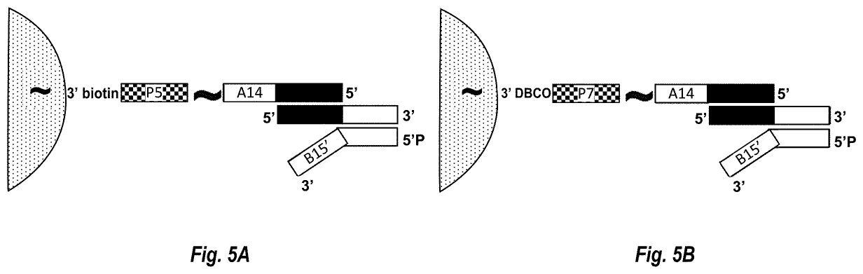

In some embodiments, nanoparticles are used for immobilization of a transposome complex. In some embodiments, the transposome complex comprises Tn5. In some embodiments, the transposome complex is immobilized by interaction of a biotin in a first transposon to an avidin on a nanoparticle (FIG. 5A). In some embodiments, the transposome complex is immobilized to the nanoparticle by a click chemistry reaction between azide and DBCO (FIG. 5B). In some embodiments, use of a click chemistry reaction improves the stability of linkage between adapter and the nanoparticles during nanoparticle and Tn5 releasing steps.

In some embodiments, the nanoparticle may immobilize a molecule that is inactive on its own, but that can serve to generate an active site. Such nanoparticles may be termed “activatable nanoparticles.” In some embodiments, nanoparticles comprise immobilized oligonucleotides that can capture transposome complexes from solution. In some embodiments, nanoparticles comprise immobilized oligonucleotides comprising a hybridization sequence that can bind to a transposon comprised in a transposome complex. In some embodiments, a nanoparticle comprises an immobilized oligonucleotide, wherein the immobilized oligonucleotide comprises a sequence for hybridizing to a hybridization sequence comprised in a second transposon comprised in a transposome complex.

Compositions comprising activatable nanoparticles have a number of advantages, such as allowing a user to control the timing of tagmentation in a multi-step method. As used herein, descriptions of nanoparticles may refer to a state of a nanoparticle wherein a transposome complex has previously been bound to an immobilized oligonucleotide comprised in the nanoparticle.

In some embodiments, the nanoparticle comprises a single immobilized transposome complex.

In some embodiments, the nanoparticle comprises more than one immobilized transposome complex. In some embodiments, the distance between each transposome complex on a nanoparticle affects the size of a library fragment produced. In some embodiments, the more than one immobilized transposome complexes are immobilized with similar distances between each transposome complex on the nanoparticle. In some embodiments, this spacing helps to generate library fragments of uniform size.

In some embodiments, the immobilized transposome complex or transposome complexes are oriented with the transposase facing away from the nanoparticle. In other words, the active domains of the transposome complex may be directed towards the outside of the composition (i.e., away from a carrier bead) to increase the probability that the transposome complex will interact with a target nucleic acid in a reaction solution.

Transposomes can be immobilized to nanoparticles in a variety of ways. In some embodiments, the transposome complex is immobilized to the nanoparticle by binding of a transposon comprising biotin, desthiobiotin, or dual biotin to avidin or streptavidin comprised on the nanoparticle. In some embodiments, the transposome complex is immobilized to the nanoparticle by a click chemistry reaction. In some embodiments, the click chemistry reaction is a reaction between an azide on the nanoparticle and a dibenzylcyclooctyne (DBCO) on the transposon. In some embodiments, an oligonucleotide capable of binding to a transposon may be immobilized in a similar manner, and a transposome complex in solution can be bound to the immobilized oligonucleotide.

In some embodiments, a carrier bead is bound to more than one nanoparticle comprising immobilized transposome complexes. In some embodiments, all the immobilized transposome complexes comprise the same first transposon. In some embodiments, all the immobilized transposome complexes are identical. In some embodiments, fragments generated by the composition incorporate the same adapter into both ends of fragments generated by the transposome complexes (i.e., symmetrical tagmentation). In some embodiments, a polynucleotide is then used to incorporate a second adapter at one end of fragments. For example, the immobilized transposome may comprise a A14 adapter sequence, while a polynucleotide may comprise a B15 adapter sequence (as shown in FIGS. 6A and 6B). As described below, methods with such transposons and polynucleotides can generate fragments that have different adapter sequences at the two ends of the produced fragments.

In some embodiments, two different transposome complexes are immobilized on nanoparticles comprised on a bead. In some embodiments, the tagmentation leads to at least some fragments that comprise a different adapter at one end of the fragment versus the other (i.e., asymmetrical tagmentation).

C. Immobilization of Nanoparticles on Carrier Beads

Nanoparticles can be immobilized on carrier beads in a number of different ways, including via electrostatic immobilization (FIG. 6A), hybridization of a clustering primer on a bead to an immobilized oligonucleotide on the surface of a nanoparticle (FIG. 6B), or a combination of these types of interactions (FIG. 6C).

Immobilization of nanoparticles on the beads can load the carrier beads with active sites comprised in the nanoparticles. For example, when the nanoparticles comprise transposome complexes, the loaded carrier beads can function similarly bead-linked transposomes (BLTs) and tagment target nucleic acid into a library of fragments. BLTs are commonly used for library preparations using tagmentation, but BLTs sometimes generate fragments with a wide range of size, including fragments that are smaller than desired.

Since steric hindrance produces spacing between transposome complexes immobilized on the nanoparticles on carrier beads (i.e., two nanoparticles cannot occupy the same surface area on a carrier bead), this spacing can prevent preparation of too-short library fragments. This advantage is intrinsic to the use of nanoparticles based on their size, and a user could use smaller or larger nanoparticles to manipulate the spacing of transposome complexes. If a user wants stricter exclusion of small fragments from a library, he/she could use nanoparticles with a larger diameter/size to avoid too-close spacing of transposome complexes. Direct loading of beads with transposome complexes (i.e., preparation of BLTs) would not have this advantage, since the small size of transposome complexes themselves could allow for immobilization of transposome complexes with too-close spacing.

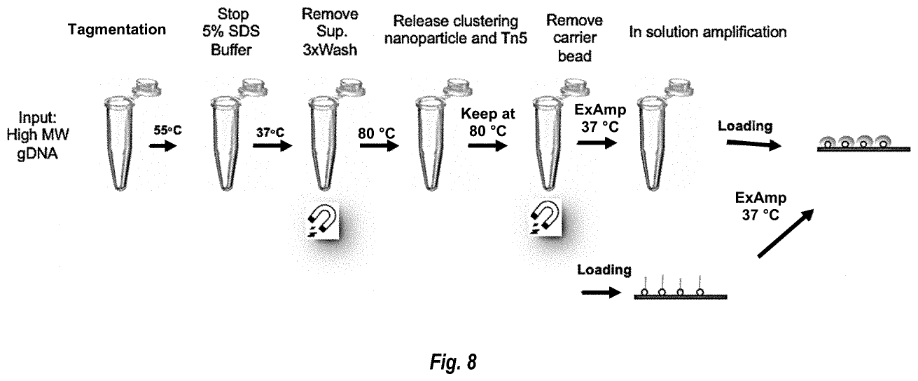

Thus, compositions with beads and at least one nanoparticle, wherein the nanoparticle comprises transposome complexes, may simplify library preparation protocols by avoiding the cost and time for size exclusion steps (such as SPRI purification) after library preparation. These compositions may also serve to provide more uniform library fragments of a desired size, as a user can calibrate the process of loading beads with nanoparticles comprising transposome complexes (as described below). These methods can include stirring of solutions with magnetic nanoparticles and/or magnetic beads to yield uniform loading of nanoparticles (as shown in FIG. 8).

In some embodiments, the interaction between the nanoparticle and carrier bead is reversible. One approach for reversible interactions is to use non-covalent interactions, such as protein-ligand (e.g., biotin streptavidin), metal-chelator (e.g., Ni-NTA-polyhistidine), or a variety of other host-guest chemistries. In some embodiments, the nanoparticles can be covalently immobilized on carrier beads with a chemically or enzymatically cleavable linker between the bead and reactive group.

In some embodiments, the linkage chemistry between the nanoparticle and the bead is alkyne azide chemistry (copper-catalyzed azide-alkyne cycloaddition chemistry). In some embodiments, the linkage chemistry is maleimide sulfhydryl chemistry.

In some embodiments, nanoparticles can be immobilized via hybridization to the clustering primers or a separate oligonucleotide can be included at a low concentration relative to the clustering primers. In some embodiments, this approach is reversible based on tuning the length of the hybridization motif such that nanoparticles can be efficiency released from carrier beads. In some embodiments, a cleavable linker can be included in the attachment to the carrier bead.

In some embodiments, a functional group for immobilizing nanoparticles on carrier beads is added via hybridization of chemically modified oligonucleotides to the sequencing primers. This approach can use any of the strategies described in approaches above and benefits from the fact that it can use existing bead surface chemistries.

D. Preparation of Compositions Comprising a Bead and at Least One Nanoparticle

In some embodiments, a method of preparing compositions may allow for multiple nanoparticles to bind to a carrier bead. In some embodiments, the number of nanoparticles bound to a carrier bead determines the size of library fragments prepared by tagmentation.

In some embodiments, the average number of nanoparticles immobilized to a bead in the mixture of compositions comprising a bead and at least one nanoparticle determines the size of target nucleic acid fragments. In some embodiments, the method does not require size selection of produced fragments before amplifying or sequencing.

In some embodiments, steric hindrance between nanoparticles comprised on the same bead reduces generation of fragments of less than 35 base pairs. In some embodiments, produced fragments are sequenced using long read sequencing as the library fragments comprise more base pairs than standard library preparation methods.

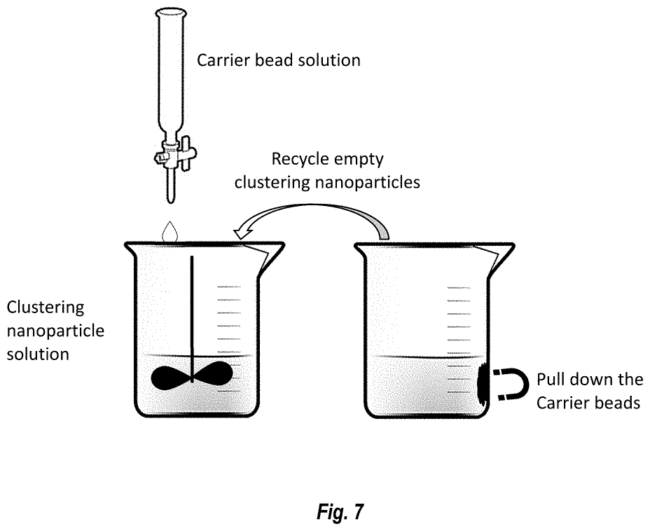

As shown in FIG. 7, the loading efficiency of nanoparticles on carrier beads may be controlled by methods of preparing compositions. In some embodiments, a carrier bead solution is added to a clustering nanoparticle solution. In some embodiments, the magnetic stir rod is used to keep carrier beads well-mixed with nanoparticles. In some embodiments, a magnetic force may be used to separate out beads to determine the average number of nanoparticles associated with each bead. In some embodiments, a user may tune the reaction to achieve a desired average number of nanoparticles associated with each bead.

In some embodiments, a method of preparing a mixture of compositions comprising a bead and at least one nanoparticle comprises (a) mixing beads and nanoparticles to prepare compositions comprising beads and nanoparticles, (b) separating the beads from the mixture, (c) evaluating the average number of nanoparticles associated with each bead, and (d) repeating prior steps until a desired average number of nanoparticles is associated with each bead in the mixture of compositions.

In some embodiments, the beads are magnetic and the mixture is performed using a magnetic field. In some embodiments, the beads are magnetic and the separating beads is performed using a magnetic field. In some embodiments, the evaluating is performed by preparing library fragments and determining fragment size.

### II. Methods of Seeding a Flow Cell

In some embodiments, fragments generated on a composition comprising a bead and at least one nanoparticle are seeded on a flow cell. In some embodiments, compositions are seeded on a flow cell while transposome complexes are immobilized. In some embodiments, fragments are released from transposome complexes and then seeded on a flow cell. In some embodiments, seeding is based on binding of an adapter sequence in fragments that binds to an oligonucleotide immobilized on the surface of a flow cell. In some embodiments, carrier beads are immobilized on a flow cell (with target nucleic acids or fragments attached to nanoparticles) by binding of a capture primer or other oligonucleotide to a flow cell.

In some embodiments, a method of seeding a flow cell comprises dissociating the bead and the nanoparticle of the composition and immobilizing the nanoparticles on the surface of the flow cell. In some embodiments, dissociating the bead and the nanoparticle is by cleavage of a cleavable linker or by dissociation of a reversible and/or non-covalent interaction between the nanoparticle and the bead. In some embodiments, a method of seeding a flow cell comprises binding the composition to the flow cell while the bead and nanoparticles are still associated with each other.

In some embodiments, a flow cell comprises a target nucleic acid or one or more fragments thereof, each bound to at least two transposome complexes immobilized on nanoparticles.

### III. Methods of Use of Compositions Comprising a Bead and at Least One Nanoparticle

In some embodiments, compositions comprising a bead and nanoparticles are used to prepare a library of fragments from a target nucleic acid. Such a method is shown in FIG. 8. In some embodiments, the target nucleic acid is genomic DNA. In some embodiments, the target nucleic acid is double-stranded DNA or a DNA:RNA duplex.

In some embodiments, after tagmentation on the composition, fragments are released from nanoparticles, amplified in solution, and amplified fragments are delivered to a flow cell for sequencing. In some embodiments, after tagmentation, fragments are delivered to a flow cell while immobilized on nanoparticles associated with a carrier bead, fragments are released and captured on a flow cell, amplified, and sequenced.

In some embodiments, amplification steps may be omitted.

In some embodiments, the methods described herein do not require a step of size selection of fragments. In some embodiments, the present methods use the loading efficiency of nanoparticles onto carrier beads to control the size of library fragments produced.

In some embodiments, a method of preparing a nucleic acid library from a target nucleic acid in a reaction solution comprises contacting the target nucleic acid with a mixture of compositions each comprising a bead and at least one nanoparticle under conditions whereby the target nucleic acid is fragmented by the transposome complexes and the 3′ transposon end sequence of the first polynucleotide is transferred to a 5′ end of the fragment, thereby producing immobilized double stranded target nucleic acid fragments wherein one strand is 5′-tagged with the tag.

In some embodiments, a method further comprises adding a sodium dodecyl sulfate (SDS) solution after the producing of fragments, wherein the SDS stops the producing of additional fragments. In some embodiments, a method further comprises releasing fragments from the transposome complexes after the producing of fragments or after adding the SDS solution. In some embodiments, the releasing is performed at a temperature of 80° C. or by amplifying.

In some embodiments, releasing the fragment from the transposome complexes also releases the fragments from the nanoparticle. In some embodiments, the fragments are in solution after the releasing.

In some embodiments, a method further comprises removing beads from the reaction solution after the releasing of fragments. In some embodiments, the beads are magnetic and removing beads is performed using a magnetic field.

In some embodiments, the beads are degradable polyester beads and removing beads is performed using a degrading agent. In some embodiments, the degrading agent is (a) a temperature of from 50° C. to 65° C., or 60° C., and/or (b) an aqueous base (as described below).

In some embodiments, fragments in solution are amplified, and the amplified fragments are loaded into a flow cell, captured, and sequenced.

In some embodiments, fragments immobilized to a mixture of compositions comprising beads and nanoparticles are loaded into a flow cell, and fragments are released and/or beads are removed, and fragments are captured on the flow cell, amplified, and sequenced. In some embodiments, fragments released from a single composition will be captured in spatial proximity on the flow cell. In some embodiments, fragments prepared on the same composition can be determined based on their spatial proximity on the flow cell. In some embodiments, fragments generated from different compositions are further apart on the flow cell as compared to fragments generated from the same composition. In some embodiments, the distance between two fragments can be used to determine whether these two fragments were likely to have been prepared on the same bead. In some embodiments, fragments prepared on the same bead were prepared from the same target nucleic acid molecule.

A. Symmetrical Tagmentation

In some embodiments, a target nucleic acid is fragmented by multiple transposome complexes, wherein all the transposome complexes are identical and the fragments are tagged with the same adapter sequence at the 5′ end of both strands of the double-stranded fragments. In some embodiments, the multiple transposome complexes are immobilized on different nanoparticles immobilized on the same carrier bead.

In some embodiments, methods using symmetrical tagmentation increases the yield of sequenceable fragments (i.e., each fragment having a different sequencing adapter sequence at each end of the fragment) as compared to standard asymmetrical tagmentation steps wherein more than one type of transposome complex is used for tagmentation. Asymmetrical tagmentation using 2 types of transposomes with different tags (A and B, such as a first-read sequencing adapter and a second-read sequencing adapter) causes loss of nearly half the reads from the amplified tagmentation products, because symmetrically and asymmetrically tagged products (A-A, B-B, A-B, B-A) are produced, but only the A-B and B-A are suitable for subsequent amplification and sequencing. In contrast, symmetrical tagmentation can increase the probability that resulting fragments will comprise both first-read and second-read sequencing adapters.

In some embodiments, methods using symmetrical tagmentation may increase yield of the library as compared to other library preparation methods.

A number of different methods for adding a second adapter after tagmentation are described herein. For example, a first-read sequencing adapter may be incorporated into double-stranded DNA or DNA:RNA duplex fragments during tagmentation, and a second-read sequencing adapter incorporated in a later step (such as by ligation). Exemplary methods will be described herein. In some embodiments, the present methods can improve library yield (compared to methods using asymmetrical tagmentation) by incorporating one sequencing adapter sequence through symmetrical tagmentation using compositions described herein and another via use of a primer or oligonucleotide comprising a second sequencing adapter sequence.

B. Polynucleotides for Incorporating One or More Adapter(s) after Tagmentation

In some embodiments, all transposomes in a composition comprising a bead and at least one nanoparticle are identical, and produced fragments comprise the same adapter sequence at both ends after tagmentation.

In some embodiments, a method using a polynucleotide is performed to incorporate an adapter sequence that is different from the adapter sequence that is incorporated by tagmentation. In some embodiments, use of a method with a polynucleotide results in fragments that have a first adapter sequence at one end and a second adapter sequence at a second end.

In some embodiments, a method comprises, after symmetrical tagmentation, releasing the double-stranded target nucleic acid fragments from the transposome complexes. In some embodiments, fragments are then immobilized to a solid support. In some embodiments, a method comprises hybridizing a polynucleotide comprising an adapter sequence and a sequence all or partially complementary to the first 3′ end transposon sequence in the released fragments, wherein the adapter sequence comprised in the polynucleotide is different from the adapter sequence comprised in the transposome complexes. In some embodiments, a second strand of the double-stranded target nucleic acid fragments is extended. In some embodiments, a polynucleotide or extended polynucleotide is ligated with the double-stranded target nucleic acid fragments to produce double-stranded fragments.

In some embodiments, the polynucleotide further comprises a unique molecular identifier (UMI) and the double-stranded target nucleic acid fragments comprise the UMI. In some embodiments, wherein the UMI is located directly adjacent to the 3′ end of the target nucleic acid fragment. In some embodiments, double stranded fragments produced are tagged with a first-read sequence adapter sequence from the first transposon at the 5′ end of one strand and with a second-read sequence adapter sequence from the polynucleotide at the 5′ end of the other strand.

In some embodiments, a method comprises, after tagmentation, releasing the double-stranded fragments from the transposome complex. In some embodiments, the fragments are immobilized to a solid support. In some embodiments, a method comprises hybridizing a first polynucleotide comprising an adapter sequence to the released fragments, wherein the adapter in the first transposon is different from the adapter in the first polynucleotide. In some embodiments, the method comprises adding a second polynucleotide comprising regions complementary to the first polynucleotide to produce a double-stranded adapter. In some embodiments, a method comprises extending a second strand of the double-stranded target nucleic acid fragments. In some embodiments, a method comprises ligating the double-stranded adapter with the double-stranded target nucleic acid fragments to produce double stranded fragments.

In some embodiments, the first polynucleotide further comprises a UMI and the double-stranded fragments comprise the UMI. In some embodiments, the UMI is located between the target nucleic acid fragment and the adapter sequence from the first polynucleotide. In some embodiments, double stranded fragments produced are tagged with a first-read sequence adapter sequence from the first transposon at the 5′ end of one strand and with a second-read sequence adapter sequence from the first polynucleotide at the 5′ end of the other strand.

C. Unique Molecular Identifiers (UMIs)

Unique molecular identifiers (UMIs) are sequences of nucleotides applied to or identified in nucleic acid molecules that may be used to distinguish individual nucleic acid molecules from one another. UMIs may be sequenced along with the nucleic acid molecules with which they are associated to determine whether the read sequences are those of one source nucleic acid molecule or another. The term “UMI” may be used herein to refer to both the sequence information of a polynucleotide and the physical polynucleotide per se. UMIs are similar to barcodes, which are commonly used to distinguish reads of one sample from reads of other samples, but UMIs are instead used to distinguish nucleic acid template fragments from another when many fragments from an individual sample are sequenced together. UMIs may be defined in many ways, such as described in WO 2019/108972 and WO 2018/136248, which are incorporated herein by reference.

In some embodiments, the library of UMIs comprises nonrandom sequences. In some embodiments, nonrandom UMIs (nrUMIs) are predefined for a particular experiment or application. In certain embodiments, rules are used to generate sequences for a set or select a sample from the set to obtain a nrUMI. For instance, the sequences of a set may be generated such that the sequences have a particular pattern or patterns. In some implementations, each sequence differs from every other sequence in the set by a particular number of (e.g., 2, 3, or 4) nucleotides. That is, no nrUMI sequence can be converted to any other available nrUMI sequence by replacing fewer than the particular number of nucleotides. In some implementations, a set of UMIs used in a sequencing process includes fewer than all possible UMIs given a particular sequence length. For instance, a set of nrUMIs having 6 nucleotides may include a total of 96 different sequences, instead of a total of 4A6=4096 possible different sequences. In some embodiments, the library of UMIs comprises 120 nonrandom sequences.

In some implementations where nrUMIs are selected from a set with fewer than all possible different sequences, the number of nrUMIs is fewer, sometimes significantly so, than the number of source DNA molecules. In such implementations, nrUMI information may be combined with other information, such as virtual UMIs, read locations on a reference sequence, and/or sequence information of reads, to identify sequence reads deriving from a same source DNA molecule.

In some embodiments, the library of UMIs may comprise random UMIs (rUMIs) that are selected as a random sample, with or without replacement, from a set of UMIs consisting of all possible different oligonucleotide sequences given one or more sequence lengths. For instance, if each UMI in the set of UMIs has n nucleotides, then the set includes 4An UMIs having sequences that are different from each other. A random sample selected from the 4An UMIs constitutes a rUMI.

In some embodiments, the library of UMIs is pseudo-random or partially random, which may comprise a mixture of nrUMIs and rUMIs.

In some embodiments, adapter sequences or other nucleotide sequences may be present between the UMI and the insert DNA.

In some embodiments, adapter sequences or other nucleotide sequences may be present between each UMI and the insert DNA.

In some embodiments, the UMI is located 3′ of the insert DNA. In some embodiments, a sequence of nucleic acids representing one or more adapter sequences may be located between the UMI and the insert DNA.

D. Linked Long Read Sequencing

Standard short read sequencing provides accurate base level sequence to provide short range information, but short read sequencing may not provide long range genomic information. Further, because haplotype information is not retained for the sequenced genome or the reference with short read data, the reconstruction of long-range haplotypes is challenging with standard methods. As such, standard sequencing and analysis approaches generally can call single nucleotide variants (SNVs), but these methods may not identify the full spectrum of structural variation seen in an individual genome. “Structural variations” of a genome, as used herein, refers to events larger than a SNV, including events of 50 base pairs or more. Representative structural variants include copy-number variations, inversions, deletions, and duplications.

“Linked long read sequencing” or “linked-read sequencing” refers to sequencing methods that provide long range information on genomic sequences.

In some embodiments, linked-read sequencing can be used for haplotype reconstruction. In some embodiments, linked-read sequencing improves calling of structural variants. In some embodiments, linked-read sequencing improves access to region of the genome with limited accessibility. In some embodiments, linked-read sequencing is used for de novo diploid assembly. In some embodiments, linked-read sequencing improves sequencing of highly polymorphic sequences (such as human leukocyte antigen genes) that require de novo assembly.

In some embodiments, linked long-read sequencing can be performed based on spatial proximity of fragments on a flow cell, wherein the fragments were generated from a given composition comprising a bead and at least one nanoparticle.

E. Linked Long-Read Sequencing Based on Spatial Separation

In some embodiments, a full-length nucleic acid is “wrapped” on a single composition comprising a bead and multiple nanoparticles, meaning that the full-length nucleic acid can associate with multiple transposome complexes immobilized on nanoparticles bound by a single carrier bead. As used herein, the nucleic acid may be DNA, cDNA, or a DNA:RNA duplex.

In some embodiments, a composition is delivered to a surface for sequencing with a full-length nucleic acid attached to the bead. The fragments could then be released, such that fragments generated from a given full-length nucleic acid (which are prepared on the same composition) would be released in close proximity, as compared to fragments prepared on other compositions.

In some embodiments, a composition is delivered to a surface for sequencing with fragments attached to the composition. In some embodiments, fragments are amplified on a flow cell after release and capture of fragments and then sequenced.

### IV. Methods of Use of Degradable Polyester Beads

In some embodiments, a degradable polyester bead comprises a plurality of transposome complexes immobilized to the surface thereof. In some embodiments, each transposome complex comprises a transposase bound to a first polynucleotide and a second polynucleotide. In some embodiments, the first polynucleotide comprises a 3′ portion comprising a transposon end sequence and a tag, and the second polynucleotide comprises a 5′ portion that is complementary to and hybridized to the transposon end sequence.

In some embodiments, the degradable polyester bead is a carrier bead. Degradable polyester beads may be used in any the embodiments of compositions comprising a bead and at least one nanoparticle described above.

In some embodiments, the polyester bead has a melting point above the temperature of steps performed in a tagmentation reaction, such as washing. In some embodiments, the polyester bead has a melting temperature of 50° C. or higher.

In some embodiments, the polyester bead has a melting point below the temperature at which a library fragment would be released from a flow cell. In some embodiments, a library fragment is associated with a flow cell based on incorporation of an adapter sequence (such as P5 (SEQ ID NO: 1) or P7 (SEQ ID NO:2) or their complements) that can hybridize to an oligonucleotide associated with the surface of a flow cell. In some embodiments, the melting temperature of a bead is below the temperature at which an adapter sequence would dehybridize from an oligonucleotide associated with the surface of a flow cell. In some embodiments, the polyester bead has a melting point of 65° C. or less.

In some embodiments, the polyester bead has a melting point of from 50° C. to 65° C. In some embodiments, the polyester bead has a melting point of 60° C.

In some embodiments, a bead comprises a target nucleic acid or one or more fragments thereof, each bound to at least two transposome complexes on the bead.

In some embodiments, degradable polyester beads may be transposome carriers. In some embodiments, degradable polyester beads may be used to mediate library preparation by tagmentation on the bead surface and allow release of the library fragments on a flow cell.

A. Degradable Polyester Beads

A “degradable polyester bead,” as used herein, can refer to any type of bead that comprises polyester and that can be degraded. In some embodiments, a degradable polyester bead may comprise a polymer. In some embodiments, polyester polymers are not cross-linked, allowing for relatively low polymer melting temperatures (such as, for example, approximately 60° C.).

Representative means for degrading a degradable polyester bead include melting by an increase in temperature or alkaline hydrolysis at an increased temperature. “Melting,” as used herein, refers to a selective depolymerization of a polyester bead by heat, such that the bead structure is lost. The de-polymerization can reduce or destroy the lattice structure of the polyester polymer. In some embodiments, melting leads to physical melting of a bead without chemical decrosslinking of the polymer. For example, melting may convert polyester beads into smaller polycaprolactone (PCL) polymers or individual molecules of PCL. In some embodiments, a PCL bead can melt at temperatures above 50° C., such that the bead degrades into smaller PCL polymers or molecules of PCL. In some embodiments, a bead melts at temperature of 50° C. to 65° C.

In some embodiments, beads may comprise polyesters other than PCL. In some embodiments, the polyester other than PCL has a melting temperature of 50° C. to 65° C. In other words, the degradable polyester bead may comprise any polyester with appropriate thermal properties. For example, the melting temperature of a degradable polyester bead is above that needed for certain reactions. For example, a degradable polyester bead may remain intact at a temperature needed to perform a tagmentation reaction, but then melt at a higher temperature to release library fragments after tagmentation for capture on a surface for sequencing.

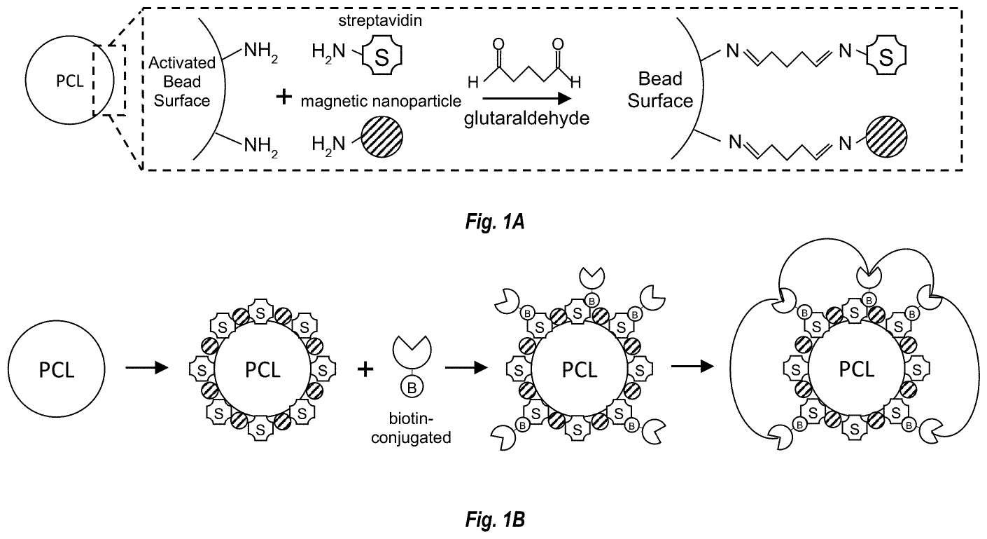

Any agents coupled to the PCL, such as streptavidin or magnetic nanoparticles (as shown in FIG. 1A) can either remain attached to smaller PCL polymers or molecules of PCL, or these agents may be released from the PCL polymers or molecules.

In some embodiments, a degradable polyester bead melts at a temperature that allows for the melting at a temperature before the temperature at which library fragments immobilized on the bead surface would dehybridize from oligonucleotide on the surface of the flow cell. In other words, a degradable polyester bead may melt at a temperature at which the library fragments are immobilized and/or remain immobilized on the flow cell. In some embodiments, a degradable polyester bead melting (while library fragments are immobilized and/or remain immobilized on its surface) allows for localized spatial release of library fragments onto a flow cell, as discussed below.

A “bead,” as used herein, is interchangeable with a microsphere. However, a bead described herein is not limited to a spherical shape. For example, a bead may be mostly spherical. A bead may be hollow, such as a spherical shell, or it may be solid. The bead may be porous, semi-porous, or nonporous. In some embodiments, the beads have limited porosity, such as being greater than 90% or greater than 95% non-porous.

In some embodiments, a nonporous bead may be solid.

In some embodiments, less than 100% of transposome complexes are on the surface of a bead. In some embodiments, a portion of transposome complexes are comprised within beads and a portion of transposome complexes are comprised on the surface of the bead. In some embodiments, all of the transposome complexes are comprised on the surface of a bead.

In some embodiments, most transposome complexes are immobilized on the surface of a polyester bead. In some embodiments, 50% or more, 60% or more, 70% or more, 80% or more, 90% or more, or 95% or more transposome complexes are immobilized on the surface of the bead. In some embodiments, having most transposomes complexes immobilized on the surface of the bead (as opposed on the inside of a bead) means that most library fragments are immobilized on the surface of the bead. In some embodiments, immobilization of most library fragments on the surface of the bead helps ensure release of library fragments from a bead in a spatially constricted area. In some embodiments, library fragments on a bead surface would be quickly released when a bead begins to melt, since surface polyester molecules would melt quickly when the reaction temperature is increased to the bead's melting temperature.

In some embodiments, degradable polyester beads are non-porous. In some embodiments, all transposome complexes are immobilized on the surface of non-porous bead, because no transposome complexes can permeabilize the bead. In some embodiments, non-porous beads do not allow for transposomes complexes to be immobilized within the bead and instead all transposomes are immobilized on the bead surface.

In some embodiments, a polyester bead may be supplied as a solid suspension, such as a 1% solid suspension or a 10 mg/ml suspension. In some embodiments, a polyester bead may be supplied as a pure solid particle.

In some embodiments, beads comprise polycaprolactone (PCL). PCL is a semicrystalline polymer known to have long-term stability, but which can be selectively degraded. For example, PCL beads may be selectively degraded by temperatures above 50° C.

In some embodiments, the polycaprolactone is poly(ε-caprolactone). In some embodiments, the beads have a diameter of 100 nm to 50 μm. In some embodiments, the beads have an average diameter of from 1 μm to 5 μm. In some embodiments, the beads have an average diameter of 3 μm to 5 μm. In some embodiments, the beads have an average diameter of 2 μm to 3 μm.

In some embodiments, the diameter of the bead has limited effect on generated library fragments. In some embodiments, the diameter of the bead does not determine the size of library fragments generated with immobilized transposome complexes. In some embodiments, library fragment size is correlated with the density of transposome complexes (comprising transposases) on the surface of degradable polyester beads used as transposomes. In some embodiments, a different amount of transposome complexes is immobilized on the surface of degradable polyester beads when different size beads are used, in order to keep the surface concentration of transposome complexes relatively constant.

In some embodiments, the PCL density is 1.145 g/cm³. In some embodiments, the PCL density is 0.75 g/cm³ to 1.5 g/cm³.

In some embodiments, a polyester bead has a melting point of from 50° C. to 65° C. In some embodiments, a polyester bead melts at a temperature of 50° C. or greater, 60° C. or greater, or 65° C. or greater. In some embodiments, a polyester bead melts at a temperature of 60° C. In some embodiments, a polyester bead degrades in the presence of an aqueous base. In some embodiments, the aqueous base is NaOH. In some embodiments, the NaOH is 1M-5M NaOH. In some embodiments, the NaOH is 3M NaOH. In some embodiments, the aqueous base is 1M, 2M, 3M, 4M, or 5M NaOH. In some embodiments, a polyester bead degrades in the presence of NaOH at a temperature of 50° C. or greater, 60° C. or greater, or 65° C. or greater.

In some embodiments, a polyester bead comprises a plurality of magnetic nanoparticles immobilized thereto.

In some embodiments, each transposome complex comprises a polynucleotide binding moiety. In some embodiments, the bead comprises a plurality of bead binding moieties covalently bound to the surface thereof. In some embodiments, the transposome complexes are immobilized to the bead surface through binding of the polynucleotide binding moieties to the bead binding moieties.

B. Transposome Complexes

In some embodiments, a plurality of transposome complexes may be immobilized to the surface of the degradable polyester beads. As used herein, the terms “immobilized” and “attached” are used interchangeably herein, and both terms are intended to encompass direct or indirect, covalent or non-covalent attachment, unless indicated otherwise, either explicitly or by context.

As used herein, a “transposome complex” refers to an integration enzyme and a nucleic acid including an integration recognition site. A transposome complex is a functional complex formed by a transposase and a transposase recognition site capable of catalyzing a transposition reaction (see, for instance, Gunderson et al., WO 2016/130704). Examples of integration enzymes include, but are not limited to, an integrase or a transposase. Examples of integration recognition sites include, but are not limited to, a transposase recognition site.

In some embodiments, degradable polyester beads comprising transposome complexes are bead-linked transposomes (BLTs) that can be used in a variety of library preparation processes. In some embodiments, the transposome complex comprises a hyperactive Tn5 transposase. BLTs comprising immobilized transposome complexes and their uses for preparing library fragments are well-known in the art, such as those described in U.S. Pat. No. 9,683,230, which is incorporated by reference in its entirety herein. In some embodiments, degradable polyester beads are comprised in a composition with at least one nanoparticle comprising immobilized transposomes, as described herein.

In some embodiments, transposome complexes are present on the bead at a density of at least 10³, 10⁴, 10⁵, 10⁶ complexes per mm². In some embodiments, the lengths of the double-stranded fragments in an immobilized library are adjusted by increasing or decreasing the density of transposome complexes present on the beads. In certain embodiments, the length of the resulting bridged fragments is less than 100 bp, 200 bp, 300 bp, 400 bp, 500 bp, 600 bp, 700 bp, 800 bp, 900 bp, 1000 bp, 1100 bp, 1200 bp, 1300 bp, 1400 bp, 1500 bp, 1600 bp, 1700 bp, 1800 bp, 1900 bp, 2000 bp, 2100 bp, 2200 bp, 2300 bp, 2400 bp, 2500 bp, 2600 bp, 2700 bp, 2800 bp, 2900 bp, 3000 bp, 3100 bp, 3200 bp, 3300 bp, 3400 bp, 3500 bp, 3600 bp, 3700 bp, 3800 bp, 3900 bp, 4000 bp, 4100 bp, 4200 bp, 4300 bp, 4400 bp, 4500 bp, 4600 bp, 4700 bp, 4800 bp, 4900 bp, 5000 bp, 10000 bp, 30000 bp or less than 100,000 bp. In some embodiments, the bridged fragments can then be amplified into clusters using standard cluster chemistry, as exemplified by the disclosure of U.S. Pat. Nos. 7,985,565 and 7,115,400, the contents of each of which is incorporated herein by reference in its entirety.

In some embodiments, the density of transposomes on the bead is controlled by the concentration of transposomes in a solution of biotin-conjugated transposomes that are added to beads during preparation of polyester beads comprising transposome complexes.

In some embodiments, each transposome complex comprises a transposase bound to a first polynucleotide and a second polynucleotide, wherein the first polynucleotide comprises a 3′ portion comprising a transposon end sequence and a tag, and the second polynucleotide comprises a 5′ portion that is complementary to and hybridized to the transposon end sequence.

In some embodiments, degradable polyester beads comprise more than one type of transposome complexes. In some embodiments, degradable polyester beads comprise two different types of transposome complexes. In some embodiments, degradable polyester beads comprising two different types of transposome complexes can produce asymmetrically tagged fragments. In some embodiments, asymmetrical tagmentation with two different transposome complexes produces some fragments with different tags at the two ends of the fragments.

In some embodiments, degradable polyester beads comprise one pool of transposome complexes comprising a tag comprising a A14 sequence and another pool of transposome complexes comprising a tag comprising a B15. In this representative example, fragments can be produced that are asymmetrically tagged with A14/B15 sequences for later PCR amplification.

In some embodiments, degradable polyester beads comprising a single type of transposome complexes (i.e., multiple identical transposomes) can produce symmetrically tagged fragments. In some embodiments, symmetrical tagmentation with two identical transposome complexes produces fragments with the same tag at both ends of the fragments. In some embodiments, methods may include steps after symmetrical tagmentation to incorporate a different adapter at one end of tagged fragments. For example, primers or polynucleotides may be used to incorporate a different adapter at one end of fragments after tagmentation. Some exemplary methods incorporating symmetrical tagmentation are described in U.S. Provisional Application No. 63/168,802, which is incorporated by reference herein in its entirety.

In some embodiments, degradable polyester beads comprise one pool of transposome complexes comprising a tag comprising a P7 sequence and another pool of transposome complexes comprising a tag comprising a P5 sequence. In this representative example, fragments can be produced that are asymmetrically tagged with sequences to bind to different capture oligonucleotides that may be present on the surface of a flow cell.

C. Tags

As used herein, a “tag” refers to a portion or domain of a polynucleotide that exhibits a sequence for a desired intended purpose or application. Some embodiments presented herein include a transposome complex comprising a polynucleotide having a 3′ portion comprising a transposon end sequence, and a tag.

Tags can comprise any sequence provided for any desired purpose. For example, in some embodiments, a tag comprises one or more restriction endonuclease recognition sites. In some embodiments, a tag comprises one or more regions suitable for hybridization with a primer for a cluster amplification reaction. In some embodiments, a tag comprises one or more regions suitable for hybridization with a primer for a sequencing reaction. It will be appreciated that any other suitable feature can be incorporated into a tag.

In some embodiments, the tag comprises a sequence having a length from 5 to 200 bp. In some embodiments, the tag comprises a sequence having a length from 10 to 100 bp. In some embodiments, the tag comprises a sequence having a length from 20 to 50 bp. In some embodiments, the tag comprises a sequence having a length of 5, 6, 7, 8, 9, 10, 20, 30, 40, 50, 60, 70, 80, 90, 100, 150 or 200 bp.

In some embodiments, a tag comprises an index sequence, a read sequencing primer sequence, an amplification primer sequence, or other type of adapter.

In some embodiments, a tag comprises an adapter. As used herein, an “adapter” refers to a linear oligonucleotide that can be fused to a nucleic acid molecule, for example, by ligation or tagmentation. In some examples, the adapter is substantially non-complementary to the 3′ end or the 5′ end of any target sequence present in the sample. In some examples, suitable adapter lengths are from 10-100 nucleotides, 12-60 nucleotides, or 15-50 nucleotides in length. Generally, the adapter can include any combination of nucleotides and/or nucleic acids. In some aspects, the adapter can include one or more cleavable groups at one or more locations. In another aspect, the adapter can include a sequence that is complementary to at least a portion of a primer, for example a primer including a universal nucleotide sequence, such as a P5 or P7 sequence. In some embodiments, an adapter comprises a P5′ or P7′ sequence. In some examples, the adapter can include a barcode (also referred to herein as an index) to assist with downstream error correction, identification, or sequencing.

In some embodiments, the tag can comprise, for example, a region for cluster amplification. In some embodiments, the tag can comprise a region for priming a sequencing reaction.

In some examples, an adapter can be modified to prevent the formation of concatemers, for example by the addition of blocking groups that prevent extension of the adapter at one or both ends. Examples of 3′ blocking groups include a 3′-spacer C3, a dideoxynucleotide, and attachment to a substrate. Examples of 5′ blocking groups include a dephosphorylated 5′ nucleotide, and attachment to a substrate.

In some examples, the adapter can include a spacer polynucleotide, which may be from 1 to 20, such as 1 to 15, or 1 to 10, nucleotides, such as 2, 3, 4, 5, 6, 7, 8, 9, or 10 nucleotides in length. In some examples, the spacer includes 10 nucleotides. In some examples, the spacer is a polyT spacer, such as a 10T spacer. Spacer nucleotides may be included at the 5′ ends of polynucleotides, which may be attached to a suitable support via a linkage with the 5′ end of the polynucleotide. Attachment can be achieved through a sulphur-containing nucleophile, such as phosphorothioate, present at the 5′ end of the polynucleotide. In some examples, the polynucleotide will include a polyT spacer and a 5′ phosphorothioate group.

In some embodiments, the adapters that are linked to the fragments of the target nucleic acid molecules include sequences for subsequent seeding, sequencing, and analysis of sequence reads pertaining to the fragments of the target nucleic acid molecules. Adapters can include, for example, capture sequences, sequencing primer binding sites, amplification primer binding sites, and indexes.

In some embodiments, an “index sequence” refers to a sequence of nucleotides that can be used as a molecular identifier and/or barcode to tag a nucleic acid, and/or to identify the source of a nucleic acid. In some examples, an index can be used to identify a single nucleic acid or a subpopulation of nucleic acids.

As used herein, a “primer” refers to a nucleic acid molecule that can hybridize to a target sequence of interest. In some embodiments, the primer functions as a substrate onto which nucleotides can be polymerized by a polymerase. In some embodiments, a primer sequence is an amplification primer sequence.

In some embodiments, an adapter includes universal nucleotide sequences for capture of the nucleic acid molecules of the sequencing library on the surface of a sequencing flow cell containing a lawn or wells having corresponding capture oligonucleotides that bind to the universal nucleotide sequence. The universal sequences present at ends of the fragments can be used for the binding of universal anchor sequences which can serve as primers and be extended in an amplification reaction. In several implementations, two different universal primers are used. One primer hybridizes with universal sequences at the 3′ end of one strand of the indexed nucleic acid fragments, and a second primer hybridizes with universal sequences at the 3′ end of the other strand of the indexed nucleic acid fragments. Thus, the anchor sequence of each primer can be different. Suitable primers can each include additional universal sequences, such as a universal capture sequence, and another index sequence. Because each primer can include an index, this step results in the addition of one or two index sequences, which can be the reverse complements of each other, or can have sequences that are not the reverse complements of each other.

In some embodiments, a tag comprises a P5 or P7 sequence, or a complement thereof. P5 and P7 may be used when referring to a universal P5 or P7 sequence or P5 or P7 primer for capture and/or amplification purposes. P5′ and P7′ designate to the complement of P5 and P7, respectively. It will be understood that any suitable universal sequence can be used in the methods presented herein, and that the use of P5 and P7 are examples only. In some embodiments, the P5 sequence comprises a sequence defined by SEQ ID NO: 1 (AATGATACGGCGACCACCGA) and the P7 sequence comprises a sequence defined by SEQ ID NO: 2 (CAAGCAGAAGACGGCATACGA). Non-limiting uses of P5 and P7 or their complements on flow cells are exemplified by the disclosures of WO 2007/010251, WO 2006/064199, WO 2005/065814, WO 2015/106941, WO 1998/044151, and WO 2000/018957, each of which is incorporated by reference herein in its entirety.

In some embodiments, a first polynucleotide comprises a tag comprising multiple different types of adapters. In some embodiments, a tag comprises 2, 3, 4, or 5 types of adapters.

D. Target Nucleic Acids

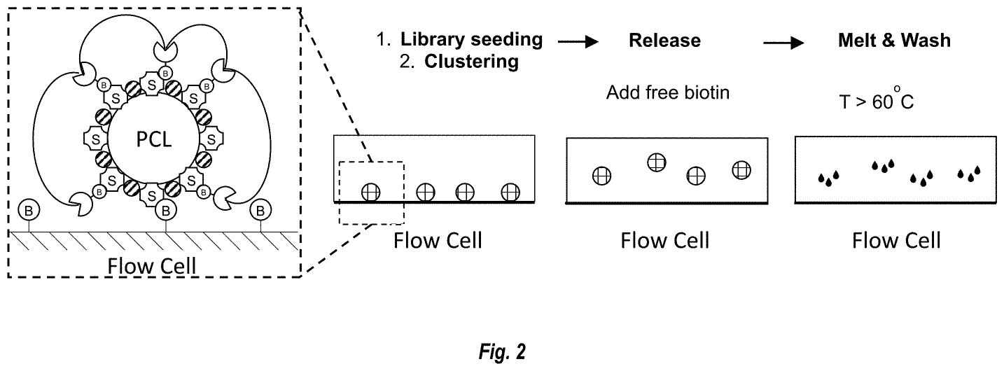

In some embodiments, a bead comprises a target nucleic acid or one or more fragments thereof, each bound to at least two transposome complexes on the bead. As shown in FIG. 2, a target nucleic acid can bind to more than one transposome complex immobilized on the surface of a bead.

A “nucleic acid,” as used herein, refers to a polymeric form of nucleotides of any length, and may include ribonucleotides, deoxyribonucleotides, analogs thereof, or mixtures thereof. The terms should be understood to include, as equivalents, analogs of either DNA or RNA made from nucleotide analogs and to be applicable to single stranded (such as sense or anti sense) and double stranded polynucleotides. The term as used herein also encompasses cDNA, that is complementary or copy DNA produced from an RNA template, for example by the action of reverse transcriptase. This term refers only to the primary structure of the molecule. Thus, the term includes triple-, double- and single-stranded DNA, as well as triple-, double- and single-stranded RNA. The terms nucleic acid molecule and polynucleotide are used interchangeably herein.

The term “target,” when used in reference to a nucleic acid molecule, is intended as a semantic identifier for the nucleic acid in the context of a method set forth herein and does not necessarily limit the structure or function of the nucleic acid beyond what is otherwise explicitly indicated.

The nucleotides in the nucleic acid molecule can include naturally occurring nucleic acids and functional analogs thereof. Particularly useful functional analogs are capable of hybridizing to a nucleic acid in a sequence specific fashion or capable of being used as a template for replication of a particular nucleotide sequence. Naturally occurring nucleic acids generally have a backbone containing phosphodiester bonds. An analog structure can have an alternate backbone linkage including any of a variety of those known in the art. Naturally occurring nucleic acids generally have a deoxyribose sugar (e.g. found in DNA) or a ribose sugar (e.g. found in RNA). A nucleic acid can contain any of a variety of analogs of these sugar moieties that are known in the art. A nucleic acid can include native or non-native bases. In this regard, a native DNA can have one or more bases selected from the group consisting of adenine, thymine, cytosine or guanine and a ribonucleic acid can have one or more bases selected from the group consisting of adenine, uracil, cytosine, or guanine. Useful non-native bases that can be included in a nucleic acid are known in the art. Examples of non-native bases include a locked nucleic acid (LNA) and a bridged nucleic acid (BNA). LNA and BNA bases can be incorporated into a DNA oligonucleotide and increase oligonucleotide hybridization strength and specificity.

Representative example biological samples from which genetic material (such as target nucleic acid molecules) can be obtained include, for example, those from a mammal such as a rodent, mouse, rat, rabbit, guinea pig, ungulate, horse, sheep, pig, goat, cow, cat, dog, primate, human or non-human primate; a plant such as Arabidopsis thaliana, corn, sorghum, oat, wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish; a reptile; an amphibian such as a frog or Xenopus laevis; a Dictyostelium discoideum; a fungi such as Pneumocystis carinii, Takifugu rubripes, yeast, Saccharamoyces cerevisiae or Schizosaccharomyces pombe; or a Plasmodium falciparum. Genetic material can also be obtained from a prokaryote such as a bacterium, Escherichia coli, staphylococci or Mycoplasma pneumoniae; an archae; a virus such as Hepatitis C virus or human immunodeficiency virus; or a viroid. Target nucleic acid molecules can be obtained from a homogeneous culture or population of the above organisms or alternatively from a collection of several different organisms, for example, in a community or ecosystem. Genetic material need not be obtained from natural sources and can instead be synthesized using known techniques.

The biological sample can be any type that comprises nucleic acid and which can be deposited onto the solid surface for tagmentation. For example, the sample can comprise DNA in a variety of states of purification, including purified DNA. However, the sample need not be completely purified, and can comprise, for example, DNA mixed with protein, other nucleic acid species, other cellular components, and/or any other contaminant.

A biological sample can comprise, for example, a crude cell lysate or whole cells. For example, a crude cell lysate that is applied to a solid support in a method set forth herein, need not have been subjected to one or more of the separation steps that are traditionally used to isolate nucleic acids from other cellular components. Exemplary separation steps are set forth in Sambrook et al., Molecular Cloning: A Laboratory Manual, 2d Edition, 1989, and Short Protocols in Molecular Biology, ed. Ausubel, et al, hereby incorporated by reference.

Thus, in some embodiments, the biological sample can comprise, for example, blood, plasma, serum, lymph, mucus, sputum, urine, semen, cerebrospinal fluid, bronchial aspirate, feces, and macerated tissue, or a lysate thereof, or any other biological specimen comprising DNA.

A sample comprising target nucleic acid molecules may be genomic DNA (for example, human genomic DNA), as well as cells and cell lysate containing target nucleic acid molecules. In some embodiments, a biological sample comprising a target nucleic acid comprises a cell lysate, whole cells, or a formalin-fixed paraffin-embedded (FFPE) tissue sample.

E. Polynucleotide Binding Moieties

In some embodiments, each transposome complex comprises a polynucleotide binding moiety. As used herein, a polynucleotide binding moiety is any moiety that allows binding of a polynucleotide to another agent. In some embodiments, a polynucleotide binding moiety serves to bind a polynucleotide to a bead.

In some embodiments, the polynucleotide binding moiety is biotin. In some embodiments, the polynucleotide binding moiety is streptavidin or avidin. In some embodiments, the polynucleotide binding moiety is biotin, and the bead binding moiety is streptavidin or avidin. In some embodiments, the bead binding moiety is biotin, and the polynucleotide binding moiety is streptavidin or avidin. In some embodiments, a polynucleotide binding moiety serves to bind a polynucleotide to a bead via binding of the polynucleotide binding moiety to the bead binding moiety.

In some embodiments, the presence of biotin as a polynucleotide binding moiety can generate a biotin-conjugated transposome.

In some embodiments, a polynucleotide binding moiety serves to immobilize one or more polynucleotide to a bead via binding of the polynucleotide binding moiety to a bead binding moiety. In some embodiments, binding of one or more polynucleotide binding moiety to a bead binding moiety serves to immobilize one or more transposome complexes to the bead. In some embodiments, one or more transposome complexes are bound to the surface of a bead.

In some embodiments, a polynucleotide binding moiety is covalently bound to a polynucleotide. In some embodiments, each polynucleotide binding moiety is covalently bound to the first polynucleotide of each transposome complex. In some embodiments, each polynucleotide binding moiety is covalently bound to the second polynucleotide of each transposome complex.

A polynucleotide binding moiety may be bound to the 5′ or 3′ of a polynucleotide. In some embodiments, a polynucleotide binding moiety is bound to the 5′ end of the first polynucleotide. In some embodiments, a polynucleotide binding moiety is bound to the 3′ end of the second polynucleotide.

F. Bead Binding Moiety

In some embodiments, a bead comprises a bead binding moiety. As used herein, a bead binding moiety is any moiety that allows binding of a bead to another agent. Beads comprising a variety of potential bead binding moieties are well-known in the art and may be commercially available.

In some embodiments, the bead binding moiety is streptavidin or avidin. In some embodiments, the bead binding moiety is biotin. In some embodiments, the bead binding moiety is streptavidin or avidin, and the polynucleotide binding moiety is biotin. In some embodiments, the bead binding moiety is biotin, and the polynucleotide binding moiety is streptavidin or avidin. In some embodiments, a polynucleotide binding moiety serves to bind a polynucleotide to a bead via binding of the polynucleotide binding moiety to the bead binding moiety.

In some embodiments, each bead binding moiety is covalently bound to the polyester bead through a linker. In some embodiments, the linker comprises —N═CH—(CH₂)₃—CH═N—, —C(O)NH—(CH₂)₆—N═, or —C(O)NH—(CH₂)₆—N═CH—(CH₂)₃CH═N—.

The present methods may use a variety of different click chemistries to prepare linkages. In some embodiments, the bead surface is functionalized in a manner that allows for different linkage chemistries. In some embodiments, the linkage chemistry is alkyne azide chemistry (copper-catalyzed azide-alkyne cycloaddition chemistry). In some embodiments, the linkage chemistry is maleimide sulfhydryl chemistry.

G. Magnetic Nanoparticles

In some embodiments, polyester beads comprise magnetic nanoparticles. In some embodiments, these magnetic particles are used for sorting and/or washing of the polyester beads. Any nanoparticles described above may used as magnetic nanoparticles with polyester beads.

In some embodiments, each magnetic nanoparticle is covalently bound to the polyester bead through a linker. In some embodiments, the linker comprises —N═CH—(CH₂)₃—CH═N—, —C(O)NH—(CH₂)₆—N═, or —C(O)NH—(CH₂)₆—N═CH—(CH₂)₃CH═N—.

In some embodiments, the magnetic nanoparticle is a bead comprising a magnetic material core. In some embodiments, the magnetic material core is iron, nickel, or cobalt. In some embodiments, the magnetic core is coated with a silica shell. In some embodiments, the silica shell allows for functionalization with organo-silane materials. In some embodiments, the magnetic nanoparticles have a diameter of 50 nm to 150 nm. In some embodiments, the magnetic nanoparticles have a diameter of 100 nm.

In some embodiments, the magnetic nanoparticles comprised in beads can be used to seed beads to multiple surfaces of a flow cell. In some embodiments, magnetic nanoparticles allow for seeding to the top and bottom surface of a sequencing surface, such as a flow cell.

In some embodiments, seeding multiple surfaces of a flow cell (e.g., top and bottom surfaces) can be performed by using beads that are immobilized on the surfaces of the flow cell. The use of beads to seed the surfaces of a flow cell allows for the creation of spatially discrete features to be formed on the surfaces of the flow cells. More specifically, a layer of beads can be formed on multiple surfaces of the flow cell such that polynucleotides present or bound to the beads are contacted or hybridized to the flow cell surface at a surface location that is closest to the bead. In this way the proximity of beads to each other in the layer determines the proximity of the polynucleotides that are hybridized or contacted on the flow cell surface. For example, polynucleotides from a tightly packed monolayer of spherical beads will produce a hybridized array that has a center-to-center spacing that is equivalent to the diameter of the beads on the flow surface(s). Accordingly, properties of the bead layer such as bead shape, bead size and bead packing density can be manipulated to obtain a desired pattern on the flow cell surface.

In some embodiments, use of magnetic beads in a “mild floating” reagent, e.g. a reagent having a density greater than 1 g/cm³ but less than 2 g/cm³, with a magnetic strip can prevent the magnetic beads sinking to the bottom too quickly. In some embodiments, approximately half the beads remain at the top surface of the flow cell while the other half of the magnetic beads sink to the bottom surface of the flow cell, as described in U.S. Provisional Application No. 63/066,727, which is incorporated herein in its entirety.

H. Immobilized Polyester Beads

In some embodiments, a polyester bead is immobilized on the surface of a flow cell.

In some embodiments, a polyester bead is immobilized on the surface of a flow cell through binding of a bead binding moiety to a flow cell binding moiety on the surface of the flow cell. In some embodiments, this binding is covalent.

In some embodiments, the polynucleotide binding moiety and the flow cell binding moiety are the same type of binding moiety. In some embodiments, the transposome complexes are bound to a first portion of the bead binding moieties on a bead and the flow cell binding moiety is bound to a second portion of the bead binding moieties on the same bead.

In some embodiments, transposome complexes may be bound to a first portion of the bead binding moieties on a bead and the flow cell binding moiety is bound to a second portion of the bead binding moieties on the same bead. For example, some bead binding moieties on a bead comprising streptavidin may bind to a biotinylated flow cell while other bead binding moieties on the same bead bind to biotinylated polynucleotides of transposome complexes, via streptavidin-biotin binding. In this way, after library seeding and clustering, the degradable polyester beads may be released from the flow cell (for example by excess free biotin) and degraded.

Thus, the degradable polyester beads can work as transposome carriers to immobilize transposomes and allow tagmentation on the surface of the bead and then bring the beads into close proximity to a flow cell. After immobilization of the bead to a flow cell, library fragments can be released to allow library seeding and clustering on the flow cell, and the polyester beads can then be degraded so as to not interfere with automation associated with library preparation, clustering, and sequencing (such sequencing by synthesis). In some embodiments, degraded polyester beads avoid clogs in tubing that may occur with non-degradable beads.

### V. Preparation of Polyester Beads Comprising Transposome Complexes

In some embodiments, a method of making a polyester bead comprising transposome complexes comprises immobilizing a plurality of transposome complexes to a polyester bead, wherein each transposome complex comprises a transposase bound to a first polynucleotide and a second polynucleotide, wherein the first polynucleotide comprises a 3′ portion comprising a transposon end sequence and a tag, and the second polynucleotide comprises a 5′ portion that is complementary to and hybridized to the transposon end sequence. In some embodiments, the method further comprises immobilizing a plurality of magnetic nanoparticles to the polyester bead.

In some embodiments, degradable polyester beads comprising transposome complexes are prepared from PCL beads. In some embodiments, the PCL beads are functionalized via introduction of active amino groups. For example, active amine groups may be introduced to the bead surface by aminolysis in a 10% (w/w) isopropanol solution in 1,6-hexanediamine. As used herein, a “functionalized bead” refers to bead with active groups on its surface.

In some embodiments, active amines on a functionalized bead are conjugated to amines on lysine residues of streptavidin to generate a streptavidin-coated bead. In some embodiments, active amines on a functionalized bead are conjugated to amine-functionalized magnetic nanoparticles by glutaraldehyde to generate a magnetic nanoparticle-coated bead. In some embodiments, a bead is coated with both streptavidin and magnetic nanoparticles. FIG. 1A provides some exemplary means to functionalize PCL beads.

Magnetic beads (i.e. beads comprising magnetic nanoparticles) have many uses within methods of bead use (See Huy et al., Faraday Discussion, 175:73-82 (2014)). For example, during wash steps, magnetic beads may be held inside a well or tube via a magnetic stand. Further, magnetic beads can be used to seed beads on the top and bottom surface of a flow cell, as discussed above.

Transposomes can be assembled onto functionalized beads in a number of ways. In an exemplary method, biotin-conjugated transposomes (such as those comprising polynucleotide binding moieties that are biotinylated) can be assembled onto PCL beads functionalized with streptavidin (FIG. 1B). PCL beads may be assembled with a single type of transposome complexes, or PCL beads may be assembled with more than one type of transposome complexes.

In some embodiments, a method of making a polyester bead comprises immobilizing a plurality of transposome complexes to a polyester bead, wherein each transposome complex comprises a transposase bound to a first polynucleotide and a second polynucleotide, wherein the first polynucleotide comprises a 3′ portion comprising a transposon end sequence and a tag, and the second polynucleotide comprises a 5′ portion that is complementary to and hybridized to the transposon end sequence.

In some embodiments, the method further comprises immobilizing a plurality of magnetic nanoparticles to the polyester bead.

In some embodiments, a method comprises immobilizing a plurality of transposome complexes to a polyester bead. In some embodiments, a method comprises immobilizing a plurality of magnetic nanoparticles to a polyester bead. In some embodiments, a method comprises immobilizing a plurality of transposome complexes and a plurality of magnetic nanoparticles to a polyester bead.

### VI. Flow Cells Comprising Polyester Beads

In some embodiments, a flow cell comprises a polyester bead described herein immobilized to the surface of the flow cell, wherein the polyester bead comprises a plurality of transposome complexes immobilized to the surface thereof, wherein each transposome complex comprises a transposase bound to a first polynucleotide and a second polynucleotide, wherein the first polynucleotide comprises a 3′ portion comprising a transposon end sequence and a tag, and the second polynucleotide comprises a 5′ portion that is complementary to and hybridized to the transposon end sequence; and wherein the polyester bead has a melting point of from 50° C. to 65° C.

In some embodiments, a polyester bead is immobilized on the surface of the flow cell through covalent binding of a bead binding moiety to a flow cell binding moiety on the surface of the flow cell. In some embodiments, the bead binding moiety is streptavidin or avidin, and the flow cell binding moiety is biotin. In some embodiments, the bead binding moiety is biotin, and the flow cell binding moiety is streptavidin or avidin.

In some embodiments, the polynucleotide binding moiety and the flow cell binding moiety are the same type of binding moiety, and the transposome complexes are bound to a first portion of the bead binding moieties on the bead and the flow cell binding moiety is bound to a second portion of the bead binding moieties on the bead.

In some embodiments, a flow cell is a sequencing flow cell. As used herein, a “sequencing flow cell” refers to a chamber comprising a surface across which one or more fluid reagents can be flowed and to which adapted fragments of sequencing libraries can transport and bind. Non-limiting examples of sequencing flow cells and related fluidic systems and detection platforms that can be readily used in the methods of the present disclosure are described, for example, in Bentley et al., Nature 456:53-59 (2008), WO 04/018497; U.S. Pat. No. 7,057,026; WO 91/06678; WO 07/123744; U.S. Pat. Nos. 7,329,492; 7,211,414; 7,315,019; 7,405,281, and US 2008/0108082, each of which is incorporated by reference herein in its entirety.

A sequencing flow cell includes a solid support having a surface on which sequencing libraries bind. In some examples, the surface contains a lawn of capture nucleotides that can bind to adapted fragments of a sequencing library. In some examples, the surface is a patterned surface. A “patterned surface” refers to an arrangement (such as an array) of different regions (such as amplification sites) in or on an exposed surface of a solid support. For example, one or more of the regions can be features where one or more amplification and/or capture primers are present. The features can be separated by interstitial regions where primers are not present. In some examples, the pattern can be an x-y format of features that are in rows and columns. In some examples, the pattern can be a repeating arrangement of features and/or interstitial regions. In some examples, the pattern can be a random arrangement of features and/or interstitial regions. In some examples, the surface is a patterned surface that contains an array of wells with capture and/or amplification nucleotides that bind to adapted fragments of a sequencing library, with interstitial regions between the wells that lack the capture and/or amplification nucleotides.

The features in a patterned surface can be wells in an array of wells (e.g. microwells or nanowells) on glass, silicon, plastic or other suitable solid supports with patterned, covalently-linked gel such as poly(N-(5-azidoacetamidylpentyl)acrylamide) (PAZAM, see, for example, US Pub. No. 2013/184796, WO 2016/066586, and WO 2015/002813, each of which is incorporated by reference herein in its entirety). The process creates gel pads used for sequencing that can be stable over sequencing runs with a large number of cycles. The covalent linking of the polymer to the wells is helpful for maintaining the gel in the structured features throughout the lifetime of the structured substrate during a variety of uses. However, in many examples the gel need not be covalently linked to the wells. For example, in some conditions silane free acrylamide (SFA, see, for example, U.S. Pat. No. 8,563,477, which is incorporated by reference herein in its entirety) which is not covalently attached to the wells of the surface, can be used as the gel material. Examples of flow cells with patterned surfaces that can be used in the methods set forth herein are described in U.S. Pat. Nos. 8,778,848, 8,778,849 and 9,079,148, and US Pub. No. 2014/0243224, each of which is incorporated by reference herein in its entirety.

The features in a patterned surface can have at any of a variety of densities including, for example, at least 10 (such as at least 100, at least 500, at least, at least 5,000, at least 10,000, at least 50,000, at least 100,000, at least 1,000,000, or at least 5,000,000, or more) features/cm².

In some examples, the flow cell device has a channel height of 50 μm, 60 μm, 70 μm, 80 μm, 90 μm, 100 μm, 110 μm, 120 μm, 130 μm, 140 μm, or 150 μm, or an amount within a range defined by any two of the aforementioned values.

In some embodiments, a solid support described herein forms at least part of a flow cell or is located in a flow cell.

The terms “solid surface,” “solid support” and other grammatical equivalents herein refer to any material that is appropriate for or can be modified to be appropriate for the attachment of materials for the processing of nucleic acids, including, for example, materials for nucleic acid library preparation, including transposome complexes. As will be appreciated by those in the art, the number of possible solid support materials is very large. Possible materials include, but are not limited to, glass and modified or functionalized glass, plastics (including acrylics, polystyrene and copolymers of styrene and other materials, polypropylene, polyethylene, polybutylene, polyurethanes, Teflon™, etc.), polysaccharides, nylon or nitrocellulose, ceramics, resins, silica or silica-based materials including silicon and modified silicon, carbon, metals, inorganic glasses, plastics, optical fiber bundles, and a variety of other polymers. Particularly useful solid supports and solid surfaces for some examples are located within a flow cell apparatus.

In some examples, a solid support includes silica-based substrates, such as glass, fused silica, or other silica-containing materials. In some examples, silica-based substrates can also be silicon, silicon dioxide, silicon nitride, or silicone hydrides. In some examples, a solid support includes plastic materials such as polyethylene, polystyrene, poly(vinyl chloride), polypropylene, nylons, polyesters, polycarbonates, cyclic olefin polymers, or poly(methyl methacrylate). In some examples, the solid support is a silica-based material or plastic material. In some examples, the solid support has at least one surface comprising glass.

In some examples, the solid support can be, or can contain, a metal. In some such examples, the metal is gold. In some examples, the solid support has at least one surface including a metal oxide. In one example, the solid support includes a tantalum oxide or tin oxide.

Acrylamide, enone, or acrylate may also be utilized as a solid support material. Other solid support materials can include, but are not limited to gallium arsenide, indium phosphide, aluminum, ceramics, polyimide, quartz, resins, polymers, and copolymers. The foregoing lists are intended to be illustrative of, but not limiting to the present application.

In some examples, the solid support and/or the solid surface can be quartz. In some examples, the solid support and/or the solid surface can be a semiconductor, such as GaAs or indium tin oxide (ITO).

Solid supports can include a single material or a plurality of different materials. Solid supports can be composites or laminates. Solid supports can be flat, round, textured and patterned. Patterns can be formed, for example, by metal pads that form features on non-metallic surfaces, for example, as described in U.S. Pat. No. 8,778,849, which is incorporated herein by reference. Another useful patterned surface is one having well features formed on a surface, for example, as described in US Pat. App. Pub. No. 2014/0243224 A1, US Pat. App. Pub. No. 2011/0172118 A1 or U.S. Pat. No. 7,622,294, each of which is incorporated herein by reference in its entirety. For examples that use a patterned surface, a gel can be associated with or deposited on the pattern features or alternatively the gel can be uniformly deposited across both the pattern features and the interstitial regions.

In some examples, the solid support comprises a patterned surface. A “patterned surface” refers to an arrangement of different regions in or on an exposed layer of a solid support. In some examples, the pattern can be an x-y format of features that are in rows and columns. In some examples, the pattern can be a repeating arrangement of features and/or interstitial regions. In some examples, the pattern can be a random arrangement of features and/or interstitial regions. Exemplary patterned surfaces that can be used in the methods and compositions set forth herein are described in U.S. Ser. No. 13/661,524 or US Pat. App. Publ. No. 2012/0316086 A1, each of which is incorporated herein by reference. Example patterned surfaces that can be used in the methods and compositions set forth herein are described in U.S. Ser. No. 13/661,524 or US Pat. App. Publ. No. 2012/0316086 A1, each of which is incorporated herein by reference.

In some examples, the solid support comprises an array of wells or depressions in a surface. This may be fabricated as is generally known in the art using a variety of techniques, including, but not limited to, photolithography, stamping techniques, molding techniques and microetching techniques. As will be appreciated by those in the art, the technique used will depend on the composition and shape of the array substrate. In some examples, the array of wells or depressions are from 10 μm to 50 μm in diameter, such as 10 μm, 15 μm, 20 μm, 25 μm, 30 μm, 35 μm, 40 μm, 45 μm, or 50 μm in diameter, or a diameter within a range defined by any two of the aforementioned values. In some examples, the wells or depressions have a depth of 0.5 μm to 1 μm, such as 0.5 μm, 0.6 μm, 0.7 μm, 0.8 μm, 0.9 μm, or 1 μm in depth, or a depth within a range defined by any two of the aforementioned values. In some examples, the wells or depressions are made of a hydrophobic material. In some examples, the hydrophobic material includes an amorphous fluoropolymer, including for example, CYTOP, Fluoropel® fluoroacrylic copolymer solution, or Teflon® fluoropolymers. See, e.g., PCT App. No. PCT/US2017/033169, which is incorporated herein by reference in its entirety.

### VII. Methods of Preparing a Nucleic Acid Library Using Degradable Beads

The polyester beads described herein can be used in methods of nucleic acid library preparation.

In some embodiments, a method of preparing a nucleic acid library from a target nucleic acid comprises contacting the target nucleic acid with a polyester bead or a flow cell described herein whereby the target nucleic acid is fragmented by the transposome complexes and the 3′ transposon end sequence of the first polynucleotide is transferred to a 5′ end of at least one strand of the fragments, thereby producing an immobilized library of fragments wherein at least one strand is 5′-tagged with the tag.

After preparation of an immobilized library of tags on the beds, the method may further comprise steps of immobilizing beads on the flow cell, releasing the library fragments (i.e., library seeding) and clustering of the library fragments on the flow cell, releasing the spend beads, and degrading the spent beads. Thus, the present method can allow bead-based preparation of a library and immobilization of the library on the flow cell using the beads as a transposome carrier, after which the beads are released and degraded. In this way, the beads serve a purpose as transposome carriers without affecting downstream methods such as sequencing.

Embodiments of the systems and methods provided herein include kits, containing any one or more of degradable polyester beads, and further including components useful for processing of the genetic material, including reagents for cell lysis, and nucleic acid amplification and sequencing, or for nucleic acid library preparation, including lysozyme, proteinase K, random hexamers, polymerase (for example, Φ29 DNA polymerase, Taq polymerase, Bsu polymerase), transposase (for example, Tn5), primers (for example, P5 and P7 adapter sequences), ligase, catalyzing enzyme, deoxynucleotide triphosphates, buffers, or divalent cations as described herein, and as used for the respective processing of genetic material.

A. Preparation of Library Fragments on the Beads

The number of steps involved to transform nucleic acids into adapter-modified templates in solution ready for cluster formation and sequencing can be reduced, or in some instances even minimized, by the use of transposase mediated fragmentation and tagging. This process, referred to herein as “tagmentation,” involves the modification of nucleic acids by a transposome complex comprising transposase enzyme complexed with polynucleotides comprising a transposon end sequence and one or more tag. Tagmentation may comprise modification of a nucleic acid molecule by a transposome complex to fragment the nucleic acid molecule and ligate adapters to the 5′ and 3′ ends of the fragments in a single step. Tagmentation may result in the simultaneous fragmentation of the DNA and ligation of the tags to the 5′ ends of both strands of duplex fragments. Tagmentation reactions can be used for preparation of sequencing libraries. Tagmentation reactions combine random fragmentation and adapter ligation into a single step to increase the efficiency of the sequencing library preparation process. In one example, following a purification step to remove the transposase enzyme, additional sequences are added to the ends of the adapted fragments by PCR. In some instances, solution-based tagmentation has drawbacks and may involve several labor-intensive steps. Additionally, bias can be introduced during PCR amplification steps.

The devices, systems, and methods presented herein overcome these drawbacks and allow unbiased sample preparation, cluster formation, and sequencing to occur on a single solid support with minimal requirements for sample manipulation or transfer, and also allow for sequencing of distinct genetic material on a solid support. In some implementations, spatial indexing of the sequencing libraries allows for simplified processing and sequence reconstruction of genetic material (for example, target nucleic acid molecules) from which sequencing libraries are generated (for example, by reducing or eliminating the need for a barcoding step). Implementations described herein also increase data resolution for sequencing of target nucleic acid molecules, and further simplify the assembly of genomes (e.g., for of new organisms), and provide improved identification of rare genetic variations and co-occurrence of mutations in target nucleic acid molecules.

In some embodiments, library fragments are retained on beads due to the association of transposases (comprised in immobilized transposome complexes on BLTs or on nanoparticles immobilized on carrier beads) with fragments. In some embodiments, fragments remain immobilized on beads until a protease or SDS is added to release fragments from the transposases or until beads are melted. In some embodiments, beads with immobilized library fragments (such as after tagmentation) are delivered to a solid support for sequencing (such as a flowcell), and the library is then released from the bead and captured on the solid support. In some embodiments, a target nucleic acid is immobilized to a bead, delivered to a solid support for sequencing, tagmentation is performed, and the library is then released from the bead and captured on the solid support. Release of library fragments from beads followed by capture on a flowcell can enable on-flowcell spatial reads, wherein fragments from a single bead will be released in close proximity to each other. In this way, fragments that are in close spatial proximity on the flowcell can be determined to have likely originated from nucleic acids that were prepared on the same bead. Such a method can be used to segregate fragments that are prepared on a given bead from fragments prepared on other beads, without the need for incorporating a “bead code” or other barcode into fragments.

In some embodiments, library fragments may be prepared by tagmentation to incorporate a bead code or other barcode, and the ability to acquire bead information based on spatial information does not preclude use of any type of barcode.

In some embodiments, preparing the sequencing libraries comprises performing a tagmentation reaction on target nucleic acid molecules bound to degradable polyester beads. As used herein, a sequencing library may comprise a collection of nucleic acid fragments of one or more target nucleic acid molecules, or amplicons of the fragments. In some embodiments, the nucleic acid fragments of the sequencing library are linked to known universal sequences (such as P5 and P7 sequences) at their 3′ and 5′ ends. In some embodiments, a sequencing library is prepared from one or more target nucleic acid molecules immobilized on a degradable polyester bead as described herein.

The adapted fragments (i.e., fragments comprised in a sequencing library) can be any appropriate size for subsequent seeding and sequencing steps. In some examples, the adapted fragments are from 150 to 400 nucleotides in length, such as from 150 to 300 nucleotides.

For example, the tagmentation reaction can be performed when the beads are captured on the sequencing flow cell or prior to loading the beads onto the sequencing flow cell. In some examples, the tagmentation reaction comprises contacting the target nucleic acid molecules with a transposomes comprising polynucleotides comprising tags comprising one or more adapter sequences.

In some embodiments, the sequencing libraries comprise DNA or RNA fragments of at least 150 nucleotides in length.

In some embodiments, gap-filling and ligation of fragments is performed using a polymerase and ligase. In some embodiments, gap-filling and ligation of fragments is performed before or after immobilizing beads to the flow cell.

B. Immobilizing Beads

In some embodiments, the method comprises immobilizing the bead comprising the immobilized library of fragments to the surface of a flow cell. In some embodiments, a bead is immobilized to the surface of a flow cell after immobilized library fragments are prepared on the bead.

In some embodiments, a bead is immobilized to the surface of the flow cell through binding of the bead binding moiety to a flow cell binding moiety on the surface of the flow cell.

C. Releasing and Capturing Library Fragments

In some embodiments, a method comprises releasing library fragments from an immobilized bead to provide a spent bead. In some embodiments, a method comprises capturing the released fragments on the flow cell surface to produce captured fragments. As used herein, a “spent bead” refers to a bead after the target nucleic acid has been fragmented and subsequently released from an immobilized bead.

In some embodiments, a degradable polyester bead may function as a solid-phase carrier, wherein target nucleic acids or library fragments are immobilized to the bead and the bead is delivered to a flow cell. WO 2015/095226 describes uses of beads as solid-phase carriers and is incorporated by reference herein in its entirety.

In some embodiments, target nucleic acids are immobilized on a bead (such as by double-stranded DNA binding to transposome complexes on the bead surface), library fragments are generated on the bead (such as by tagmentation), the bead is delivered to a flow cell, and the library fragments are released and captured on the flow cell. Alternatively, target nucleic acids can be immobilized to the bead, the bead is delivered to a flow cell, library fragments are generating on the bead, and library fragments are released and captured on the flow cell.

In some embodiments, fragments are released from beads before the beads are released from the flow cell. In some embodiments, immobilized library fragments are generated on beads (wherein the fragments are generated either before or after beads are immobilized on a flow cell), fragments are released from the beads, fragments are captured on the flow cell, and the beads are then released from the flow cell and degraded. In some embodiments, a detergent or surfactant is used to release library fragments. In some embodiments, SDS is used to release library fragments. In some embodiments, bead purification removes a transposase and releases library fragments. In some embodiments, melting of the bead releases library fragments, which are then captured by the flow cell.

In some embodiments, capturing the released library fragments comprises hybridizing the released fragments to capture oligonucleotides on the surface of the flow cell. In some embodiments, after release, the sequencing libraries transport to the surface of the flow cell, where they are captured. Then, seeding on the flow cell with sequencing library fragments from a single bead occurs in close proximity to where the bead is bound to the flow cell. Because seeding occurs in close proximity to the footprint on the flow cell of each bead, the seeded sequencing library from each bead is spatially segregated (or “indexed”) on the flow cell based on the location of the bead.

As used herein, “capture” refers to immobilization of a target entity (such as a polyester bead) on a surface of interest (such as a flow cell surface). A capture site is a site on a surface of a sequencing flow cell where one or more beads or adapted fragments of a target nucleic acid molecule can be captured. “Capture oligonucleotides,” as used herein, refer to a nucleic acid that is complementary to at least a portion of library fragments. In some embodiments, capture oligonucleotides comprise primer sequences and may be referred to as “capture primers.”

In some examples, the sequencing libraries are captured on the flow cell by the interaction of a capture oligonucleotide on the flow cell with the adapted fragments of the sequencing libraries.

In some examples, the capture oligonucleotide is a first member of a specific binding pair that is located on the sequencing flow cell and binds to a second member of the specific binding pair located on the adapted fragments (i.e., fragments generated by tagmentation) of the sequencing library. For example, the flow cell may be functionalized with a first member of a specific binding pair and the adapters of the adapted fragments contain the second member of the specific binding pair.

In some examples, the capture oligonucleotide can be attached to the surface of the sequencing flow cell. For example, the capture oligonucleotide can be attached to wells on the surface of a patterned flow cell. The attachment can be via an intermediate structure such as a bead, particle, or gel. Attachment of capture oligonucleotides to the surface of a sequencing flow cell via a gel is exemplified by flow cells available commercially from Illumina Inc. (San Diego, Calif.) or described in WO 2008/093098, which is incorporated herein by reference in its entirety.

In some embodiments, a patterned flow cell contains a surface for binding to sequencing libraries made by patterning a solid support material with wells (e.g. microwells or nanowells), coating the patterned support with a gel material (e.g. PAZAM, SFA or chemically modified variants thereof, such as the azidolyzed version of SFA (azido-SFA)) and polishing the gel coated support, for example via chemical or mechanical polishing, thereby retaining gel in the wells but removing or inactivating substantially all of the gel from the interstitial regions on the surface of the structured substrate between the wells. Capture oligonucleotides can be attached to gel material for capture and amplification of the sequencing libraries. The sequencing libraries can then be transported to the patterned surface such that individual adapted fragments in the libraries will seed individual wells via interactions with primers attached to the gel material; however, the adapted fragments will not occupy the interstitial regions between wells due to absence or inactivity of the gel material. Amplification of the adapted fragments will be confined to the wells since absence or inactivity of gel in the interstitial regions between wells prevents outward migration of the growing nucleic acid colony. The process can be conveniently manufactured, being scalable and utilizing conventional micro- or nanofabrication methods.

In some embodiments, a capture oligonucleotide may comprise a universal nucleotide sequence. A universal nucleotide sequence, as used here refers to a region of sequence that is common to two or more nucleic acid molecules where the molecules also have regions of sequence that differ from each other. A universal sequence that is present in different members of a collection of molecules can allow capture of multiple different nucleic acids using a population of universal capture nucleic acids, e.g., capture oligonucleotides that are complementary to a portion of the universal sequence, e.g., a universal capture sequence. Non-limiting examples of universal capture sequences include sequences that are identical to or complementary to P5 and P7 primers. Similarly, a universal sequence present in different members of a collection of molecules can allow the amplification or replication (e.g., sequencing) of multiple different nucleic acids using a population of universal primers that are complementary to a portion of the universal sequence, e.g., a universal anchor sequence. A capture oligonucleotide or a universal primer therefore includes a sequence that can hybridize specifically to a universal sequence. Two universal sequences that hybridize are referred to as a universal binding pair. For instance, a capture oligonucleotide and a universal capture sequence that hybridize are a universal binding pair.

As used herein, seeding a sequencing library refers to immobilization of adapted fragments of a target nucleic acid molecule on a solid support, such as a sequencing flow cell.

D. Amplifying Fragments

In some embodiments, the method comprises amplifying the fragments off the bead.

In some examples, the seeded sequencing libraries may be amplified prior to sequencing. For example, the seeded sequencing libraries may be amplified using primer sites in the adapter sequences, and subsequently sequenced using sequencing primer sites in the adapter sequences in one or more tag.

In some embodiments, the target nucleic acid molecules are genomic DNA and the amplification involves whole-genome amplification.

In some embodiments, a method comprises amplifying the captured fragments on the flow cell surface to produce immobilized, amplified fragments. In some embodiments, amplifying the captured fragments comprises bridge amplification to produce clusters of the fragments.

As used herein, “amplification” refers to an action or process whereby at least a portion of a nucleic acid molecule is replicated or copied into at least one additional nucleic acid molecule. In some examples, such amplification can be performed using isothermal conditions; in other examples, such amplification can include thermocycling. In some examples, the amplification is a multiplex amplification that includes the simultaneous amplification of a plurality of target sequences in a single amplification reaction. Non-limiting examples of amplification reactions include polymerase chain reaction (PCR), ligase chain reactions, strand displacement amplification reaction (SDA), rolling circle amplification reaction (RCA), multiple annealing and looping based amplification cycles (MALBAC), transcription-mediated amplification (TMA) methods such as NASBA, loop mediated amplification methods (e.g., “LAMP” amplification using loop-forming sequences. The nucleic acid molecule that is amplified can be DNA comprising, consisting of, or derived from DNA or ribonucleic acid (RNA) or a mixture of DNA and RNA, including modified DNA and/or RNA. The products resulting from amplification of a nucleic acid molecule or molecules (for example, “amplification products” or “amplicons”), whether the starting nucleic acid is DNA, RNA or both, can be either DNA or RNA, or a mixture of both DNA and RNA nucleosides or nucleotides, or they can comprise modified DNA or RNA nucleosides or nucleotides. A “copy” does not necessarily mean perfect sequence complementarity or identity to the target sequence. For example, copies can include nucleotide analogs such as deoxyinosine or deoxyuridine, intentional sequence alterations (such as sequence alterations introduced through a primer comprising a sequence that is hybridizable, but not complementary, to the target sequence), and/or sequence errors that occur during amplification.

Several examples include solid-phase amplification, which is an amplification reaction carried out on or in association with a solid support such that all or a portion of the amplified products are immobilized on the solid support as they are formed. Non-limiting examples of solid-phase amplification include solid-phase polymerase chain reaction (solid-phase PCR) and solid phase isothermal amplification, which are reactions analogous to standard solution phase amplification, except that one or both of the forward and reverse amplification primers is/are immobilized on the solid support.

In additional examples, the amplification can include, but is not limited to, the PCR, SDA, TMA, and nucleic acid sequence-based amplification (NASBA), as described in U.S. Pat. No. 8,003,354, which is incorporated herein by reference in its entirety. The above amplification methods can be employed to amplify one or more nucleic acids of interest. For example, PCR, including multiplex PCR, SDA, TMA, NASBA and the like can be utilized to amplify encapsulated nucleic acids. In some examples, primers directed specifically to the nucleic acid of interest are included in the amplification reaction.

In some examples, the amplification method can include ligation probe amplification or oligonucleotide ligation assay (OLA) reactions that contain primers directed specifically to the nucleic acid of interest. In some examples, the amplification method can include a primer extension-ligation reaction that contains primers directed specifically to the nucleic acid of interest. As a non-limiting example of primer extension and ligation primers that can be specifically designed to amplify a nucleic acid of interest, the amplification can include primers used for the GoldenGate assay (Illumina, Inc., San Diego, Calif.) as exemplified by U.S. Pat. Nos. 7,582,420 and 7,611,869, each of which is incorporated herein by reference in its entirety.

Another nucleic acid amplification method that is useful in the present disclosure is tagged PCR which uses a population of two-domain primers having a constant 5′ region followed by a random 3′ region as described, for example, in Grothues et al. Nucleic Acids Res. 21(5): 1321-2 (1993), incorporated herein by reference in its entirety. The first rounds of amplification are carried out to allow a multitude of initiations on heat denatured DNA based on individual hybridization from the randomly-synthesized 3′ region. Due to the nature of the 3′ region, the sites of initiation are contemplated to be random throughout the genome. Thereafter, the unbound primers can be removed and further replication can take place using primers complementary to the constant 5′ region.

In some examples, the seeded sequencing libraries are amplified by solid-phase amplification. Primers (such as capture primers) for solid-phase amplification may be immobilized by single point covalent attachment to the solid support at or near the 5′ end of the primer, leaving the template-specific portion of the primer free to anneal to its cognate template and the 3′ hydroxyl group free for primer extension. Any suitable covalent attachment means may be used for this purpose. The chosen attachment chemistry will depend on the nature of the solid support, and any derivatization or functionalization applied to it. The primer itself may include a moiety, which may be a non-nucleotide chemical modification, to facilitate attachment. In a particular example, the primer may include a sulphur-containing nucleophile, such as phosphorothioate or thiophosphate, at the 5′ end. In the case of solid-supported polyacrylamide hydrogels, this nucleophile will bind to a bromoacetamide group present in the hydrogel. A more particular means of attaching primers and templates to a solid support is via 5′ phosphorothioate attachment to a hydrogel comprised of polymerized acrylamide and N-(5-bromoacetamidylpentyl) acrylamide (BRAPA), as described in WO 05/065814, which is incorporated by reference herein in its entirety.

Although the disclosure encompasses solid-phase amplification methods in which only one amplification primer is immobilized (the other primer usually being present in free solution), in some examples the solid support may be provided with both the forward and the reverse primers immobilized. In practice, there will be a ‘plurality’ of identical forward primers and/or a ‘plurality’ of identical reverse primers immobilized on the solid support, since the amplification process uses an excess of primers to sustain amplification. References herein to forward and reverse primers are to be interpreted accordingly as encompassing a ‘plurality’ of such primers unless the context indicates otherwise.

The surface of the sequencing flow cell can include a plurality of primers that are used to produce amplicons from a sequencing library seeded on the flow cell. In some examples, the primers can have a universal priming sequence that is complementary to a universal sequence that is present in an adapter sequence ligated to of each target nucleic acid. In particular examples, the plurality of primers can be attached to the amplification site. The primers can be attached to an amplification site as set forth above for capture nucleic acids.

In some examples, the seeded sequencing libraries can be amplified using cluster amplification methodologies as exemplified by the disclosures of U.S. Pat. Nos. 7,985,565 and 7,115,400, the contents of each of which are incorporated herein by reference in their entirety. The incorporated materials of U.S. Pat. Nos. 7,985,565 and 7,115,400 describe methods of nucleic acid amplification which allow amplification products to be immobilized on a solid support in order to form arrays comprised of clusters or “colonies” of immobilized nucleic acid molecules. Each cluster or colony on such an array is formed from a plurality of identical or substantially identical immobilized polynucleotide strands and a plurality of identical immobilized complementary polynucleotide strands. The arrays so-formed are generally referred to herein as “clustered arrays”. The products of solid-phase amplification reactions such as those described in U.S. Pat. Nos. 7,985,565 and 7,115,400 are so-called “bridged” structures formed by annealing of pairs of immobilized polynucleotide strands and immobilized complementary strands, both strands being immobilized on the solid support at the 5′ end, in some cases via a covalent attachment. Cluster amplification methodologies are examples of methods wherein an immobilized nucleic acid template is used to produce immobilized amplicons.

It will be appreciated that a small amount of contamination can be present in a colony or cluster without negatively affecting a subsequent sequencing reaction. Example levels of contamination that can be acceptable at an individual amplification site for particular applications include, but are not limited to, at most 0.1%, 0.5%, 1%, 5%, 10% or 25% contaminating amplicons.

Other suitable methodologies can also be used to produce immobilized amplicons from immobilized DNA fragments produced according to the methods provided herein. For example, one or more clusters or colonies can be formed via solid-phase PCR whether one or both primers of each pair of amplification primers are immobilized. In some examples, the encapsulated nucleic acids are amplified within the beads, and then deposited in an array or on a solid support in a cluster.

E. Releasing Beads

In some embodiments, a method comprises detaching the spent bead from the flow cell surface by treating the spent bead with an excess of solution-phase flow cell binding moiety to provide a solution-phase spent bead. Free biotin can complete for binding to streptavidin on the surface of the beads and allow release of beads from the biotin-conjugated flow cell.

F. Degrading Beads

In some embodiments, a method comprises degrading a solution-phase spent bead with a degrading agent. In some embodiments, a method comprises removing the degraded bead from the flow cell. As used herein, a “degraded bead” refers to a bead that has been broken down (such as by depolymerization at a temperature above 50° C.). Generally, a degraded bead may be a spent bead from which library fragments has been released and that has then been degraded.

The present method does not require that all beads release library fragments before being degraded, since some loss of library product would be acceptable. In some embodiments, a majority of beads have released immobilized library fragments before the beads are degraded. In some embodiments, at least 50%, at least 60%, at least 70%, at least 80%, at least 90%, at least 95%, or at least 99% of beads have been spent (i.e., library fragments have been released) before the beads are degraded.

In some embodiments, polyester from beads that are degraded mixes with reaction buffers and is removed from a flow cell. In some embodiments, the degraded polyester exits the flow cell through tubing without impeding buffer flow through the tubing. In some embodiments, mixing of degraded polyester with buffer causes a relatively uniform density of polyester in the buffer solution, such that this density does not impede buffer flow through tubing.

In some embodiments, degrading of beads decreases the chance of bead clumping, wherein bead clumping refers to beads being in association with each other or randomly in close proximity. In some embodiments, reducing bead clumping also reduces blockage of tubing or flow cells by beads.

In some embodiments, non-degraded beads may settle in tubing or in flow cells due to their higher density than the buffer, while polyester from degraded beads does not settle (since the polyester from degraded beads is in a relatively uniform density within the buffer).

Further, the present method does not require that all beads are degraded by the method. A relatively small fraction of beads will not likely impact downstream processes and/or increase the risk of clogging during solution changes. In other words, degrading a portion of the beads used as transposome carriers improves the present method compared to method using beads that do not degrade at all. In some embodiments, a majority of beads are degraded. In some embodiments, at least 50%, at least 60%, at least 70%, at least 80%, at least 90%, at least 90%, or at least 99% of beads are degraded with a degrading agent.

In some embodiments, the degrading agent is a temperature of from 50° C. to 65° C. In some embodiments, the degrading agent is a temperature of greater than 50° C., greater than 60° C., or greater than 65° C. In some embodiments, the degrading agent is a temperature of 60° C.

In some embodiments, the degrading agent is an aqueous base. In some embodiments, the aqueous base is NaOH. In some embodiments, the NaOH is 1M-5M NaOH. In some embodiments, the NaOH is 3M NaOH (See Yeo et al., J Biomed Mater Res B Appl Biomater 87(2):562-9 (2008)).

In some embodiments, the degrading agent comprises both aqueous base and a temperature of from 50° C. to 65° C. In some embodiments, the degrading agent comprises aqueous NaOH at a temperature of from 50° C. to 65° C. In some embodiments, the degrading agent comprises aqueous NaOH at a temperature of greater than 50° C., greater than 60° C., or greater than 65° C. In some embodiments, the degrading agent comprises aqueous NaOH at a temperature of 60° C.

In some embodiments, the method comprises removing the degraded bead from the flow cell. In some embodiments, a wash step removes degraded beads from a flow cell.

G. Sequencing

In some embodiments, a method comprises sequencing the immobilized, amplified fragments or the clusters of the fragments.

In some embodiments, sequencing libraries are not barcoded to identify individual beads. In some embodiments, the method further comprises sequencing the sequencing libraries seeded on the surface of the flow cell. In some examples, the location on the surface of the flow cell of sequencing libraries seeded from respective degradable polyester beads is used as a spatial index for reads generated from the sequencing of the sequence libraries.

In some embodiments, the seeded sequencing libraries are sequenced in full or in part. The seeded sequencing libraries can be sequenced according to any suitable sequencing methodology, such as direct sequencing, including SBS, sequencing by ligation, sequencing by hybridization, nanopore sequencing and the like. Non-limiting examples of methods for determining the sequence of immobilized nucleic acid fragments are described, for instance, in Bignell et al. (U.S. Pat. No. 8,053,192), Gunderson et al. (WO2016/130704), Shen et al. (U.S. Pat. No. 8,895,249), and Pipenburg et al. (U.S. Pat. No. 9,309,502), each of which is incorporated by reference herein in its entirety.

The methods described herein can be used in conjunction with a variety of nucleic acid sequencing techniques. Particularly applicable techniques are those wherein nucleic acids are attached at fixed locations in an array such that their relative positions do not change and wherein the array is repeatedly imaged. Implementations in which images are obtained in different color channels, for example, coinciding with different labels used to distinguish one nucleotide base type from another are particularly applicable. In some examples, the process to determine the nucleotide sequence of a fragment can be an automated process.

One sequencing methodology is SBS. In SBS, extension of a nucleic acid primer along a nucleic acid template (e.g. a target nucleic acid or amplicon thereof) is monitored to determine the sequence of nucleotides in the template. The underlying chemical process can be polymerization (e.g. as catalyzed by a polymerase enzyme). In some polymerase-based SBS implementations, fluorescently labeled nucleotides are added to a primer (thereby extending the primer) in a template dependent fashion such that detection of the order and type of nucleotides added to the primer can be used to determine the sequence of the template.

In one example, a nucleotide monomer includes locked nucleic acids (LNAs) or bridged nucleic acids (BNAs). The use of LNAs or BNAs in a nucleotide monomer increases hybridization strength between a nucleotide monomer and a sequencing primer sequence present on an immobilized fragment.

SBS can use nucleotide monomers that have a terminator moiety or those that lack any terminator moieties. Methods using nucleotide monomers lacking terminators include, for example, pyrosequencing and sequencing using γ-phosphate-labeled nucleotides, as set forth in further detail herein. In methods using nucleotide monomers lacking terminators, the number of nucleotides added in each cycle is generally variable and dependent upon the template sequence and the mode of nucleotide delivery. For SBS techniques that utilize nucleotide monomers having a terminator moiety, the terminator can be effectively irreversible under the sequencing conditions used as is the case for traditional Sanger sequencing which utilizes dideoxy nucleotides, or the terminator can be reversible as is the case for sequencing methods developed by Solexa (now Illumina, Inc.).

SBS techniques can use nucleotide monomers that have a label moiety or those that lack a label moiety. Accordingly, incorporation events can be detected based on a characteristic of the label, such as fluorescence of the label; a characteristic of the nucleotide monomer such as molecular weight or charge; a byproduct of incorporation of the nucleotide, such as release of pyrophosphate; or the like. In embodiments, where two or more different nucleotides are present in a sequencing reagent, the different nucleotides can be distinguishable from each other, or alternatively the two or more different labels can be the indistinguishable under the detection techniques being used. For example, the different nucleotides present in a sequencing reagent can have different labels and they can be distinguished using appropriate optics as exemplified by the sequencing methods developed by Solexa (now Illumina, Inc.).

Some examples include pyrosequencing techniques. Pyrosequencing detects the release of inorganic pyrophosphate (PPi) as particular nucleotides are incorporated into the nascent strand (Ronaghi et al., Analytical Biochemistry, 242(1):84-9, 1996, Ronaghi, Genome Res., 11(1):3-11, 2001, Ronaghi, Uhlen, and Nyren, Science, 281(5375), 363, 1998, and U.S. Pat. Nos. 6,210,891; 6,258,568 and 6,274,320, each of which is incorporated by reference herein in its entirety). In pyrosequencing, released PPi can be detected by being immediately converted to adenosine triphosphate (ATP) by ATP sulfurase, and the level of ATP generated is detected via luciferase-produced photons. The nucleic acids to be sequenced can be attached to features in an array and the array can be imaged to capture the chemiluminescent signals that are produced due to incorporation of a nucleotides at the features of the array. An image can be obtained after the array is treated with a particular nucleotide type (e.g. A, T, C, or G). Images obtained after addition of each nucleotide type will differ with regard to which features in the array are detected. These differences in the image reflect the different sequence content of the features on the array. However, the relative locations of each feature will remain unchanged in the images. The images can be stored, processed, and analyzed using the methods set forth herein. For example, images obtained after treatment of the array with each different nucleotide type can be handled in the same way as exemplified herein for images obtained from different detection channels for reversible terminator-based sequencing methods.

In another example type of SBS, cycle sequencing is accomplished by stepwise addition of reversible terminator nucleotides containing, for example, a cleavable or photobleachable dye label as described, for example, in WO 04/018497, WO 91/06678, WO 07/123,744, and U.S. Pat. No. 7,057,026, each of which is incorporated by reference herein in its entirety. The availability of fluorescently-labeled terminators in which both the termination can be reversed and the fluorescent label cleaved facilitates efficient cyclic reversible termination (CRT) sequencing. Polymerases can also be co-engineered to efficiently incorporate and extend from these modified nucleotides.

In some reversible terminator-based sequencing embodiments, the labels do not substantially inhibit extension under SBS reaction conditions. However, the detection labels can be removable, for example, by cleavage or degradation. Images can be captured following incorporation of labels into arrayed nucleic acid features. In particular examples, each cycle involves simultaneous delivery of four different nucleotide types to the array and each nucleotide type has a spectrally distinct label. Four images can then be obtained, each using a detection channel that is selective for one of the four different labels. Alternatively, different nucleotide types can be added sequentially and an image of the array can be obtained between each addition step. In such examples, each image will show nucleic acid features that have incorporated nucleotides of a particular type. Different features will be present or absent in the different images due the different sequence content of each feature. However, the relative position of the features will remain unchanged in the images. Images obtained from such reversible terminator—SBS methods can be stored, processed, and analyzed as set forth herein. Following the image capture step, labels can be removed and reversible terminator moieties can be removed for subsequent cycles of nucleotide addition and detection. Removal of the labels after they have been detected in a particular cycle and prior to a subsequent cycle can provide the advantage of reducing background signal and crosstalk between cycles. Examples of useful labels and removal methods are set forth herein.

In some embodiments, some or all of the nucleotide monomers can include reversible terminators. In some embodiments, reversible terminators/cleavable fluorophores can include fluorophores linked to the ribose moiety via a 3′ ester linkage (see, e.g., Metzker, Genome Res., 15:1767-1776, 2005, which is incorporated by reference herein in its entirety). Other approaches have separated the terminator chemistry from the cleavage of the fluorescence label (see, e.g., Ruparel et al., Proc Natl Acad Sci USA 102: 5932-7, 2005, which is incorporated by reference herein in its entirety). Ruparel et al. described the development of reversible terminators that used a small 3′ allyl group to block extension, but could easily be deblocked by a short treatment with a palladium catalyst. The fluorophore was attached to the base via a photocleavable linker that could easily be cleaved by a 30 second exposure to long wavelength UV light. Thus, either disulfide reduction or photocleavage can be used as a cleavable linker. Another approach to reversible termination is the use of natural termination that ensues after placement of a bulky dye on a dNTP. The presence of a charged bulky dye on the dNTP can act as an effective terminator through steric and/or electrostatic hindrance. The presence of one incorporation event prevents further incorporations unless the dye is removed. Cleavage of the dye removes the fluorophore and effectively reverses the termination. Examples of modified nucleotides are also described in U.S. Pat. Nos. 7,427,673, and 7,057,026, each of which is incorporated by reference herein in its entirety.

Additional example SBS systems and methods which can be utilized with the methods and systems described herein are described in US Pub. Nos. 2007/0166705, 2006/0188901, 2006/0240439, 2006/0281109, 2012/0270305, and 2013/0260372, U.S. Pat. No. 7,057,026, PCT Publication No. WO 05/065814, US Patent Application Publication No. 2005/0100900, and PCT Publication Nos. WO 06/064199 and WO 07/010,251, each of which is incorporated by reference herein in its entirety.

Some examples use detection of four different nucleotides using fewer than four different labels. For example, SBS can be performed using methods and systems described in US Pub. No. 2013/0079232, which is incorporated by reference herein in its entirety. As a first example, a pair of nucleotide types can be detected at the same wavelength, but distinguished based on a difference in intensity for one member of the pair compared to the other, or based on a change to one member of the pair (e.g. via chemical modification, photochemical modification or physical modification) that causes apparent signal to appear or disappear compared to the signal detected for the other member of the pair. As a second example, three of four different nucleotide types can be detected under particular conditions while a fourth nucleotide type lacks a label that is detectable under those conditions, or is minimally detected under those conditions (e.g., minimal detection due to background fluorescence, etc.). Incorporation of the first three nucleotide types into a nucleic acid can be determined based on presence of their respective signals and incorporation of the fourth nucleotide type into the nucleic acid can be determined based on absence or minimal detection of any signal. As a third example, one nucleotide type can include label(s) that are detected in two different channels, whereas other nucleotide types are detected in no more than one of the channels. The aforementioned three example configurations are not considered mutually exclusive and can be used in various combinations. An example that combines all three examples, is a fluorescent-based SBS method that uses a first nucleotide type that is detected in a first channel (e.g. dATP having a label that is detected in the first channel when excited by a first excitation wavelength), a second nucleotide type that is detected in a second channel (e.g. dCTP having a label that is detected in the second channel when excited by a second excitation wavelength), a third nucleotide type that is detected in both the first and the second channel (e.g. dTTP having at least one label that is detected in both channels when excited by the first and/or second excitation wavelength) and a fourth nucleotide type that lacks a label that is not, or minimally, detected in either channel (e.g. dGTP having no label).

Further, as described in the incorporated materials of US Pub. No. 2013/0079232, sequencing data can be obtained using a single channel. In such so-called one-dye sequencing approaches, the first nucleotide type is labeled but the label is removed after the first image is generated, and the second nucleotide type is labeled only after a first image is generated. The third nucleotide type retains its label in both the first and second images, and the fourth nucleotide type remains unlabeled in both images.

Some examples can use sequencing by ligation techniques. Such techniques use DNA ligase to incorporate oligonucleotides and identify the incorporation of such oligonucleotides. In several implementations, the oligonucleotides have different labels that are correlated with the identity of a particular nucleotide in a sequence to which the oligonucleotides hybridize. As with other SBS methods, images can be obtained following treatment of an array of nucleic acid features with the labeled sequencing reagents. Each image will show nucleic acid features that have incorporated labels of a particular type. Different features will be present or absent in the different images due the different sequence content of each feature, but the relative position of the features will remain unchanged in the images. Images obtained from ligation-based sequencing methods can be stored, processed, and analyzed as set forth herein. Example SBS systems and methods which can be utilized with the methods and systems described herein are described in U.S. Pat. Nos. 6,969,488, 6,172,218, and 6,306,597.

Some examples can use nanopore sequencing (see, e.g., Deamer and Akeson, Trends Biotechnol., 18, 147-151, 2000, Deamer and Branton, Acc. Chem. Res., 35:817-825, 2002, Li, Gershow, Stein, Brandin, and Golovchenko, Nat. Mater., 2:611-615, 2003, each of which is incorporated by reference herein in its entirety). In such examples, the fragment passes through a nanopore. The nanopore can be a synthetic pore or biological membrane protein, such as α-hemolysin. As the fragment passes through the nanopore, each base-pair can be identified by measuring fluctuations in the electrical conductance of the pore (see, e.g., U.S. Pat. No. 7,001,792; Soni and Meller, Clin. Chem. 53, 1996-2001, 2007; Healy, Nanomed., 2, 459-481, 2007; and Cockroft et al., J. Am. Chem. Soc., 130, 818-820, 2008, each of which is incorporated by reference herein in its entirety). Data obtained from nanopore sequencing can be stored, processed, and analyzed as set forth herein. In particular, the data can be treated as an image in accordance with the example treatment of optical images and other images that is set forth herein.

In some embodiments, methods involve the real-time monitoring of DNA polymerase activity. Nucleotide incorporations can be detected through fluorescence resonance energy transfer (FRET) interactions between a fluorophore-bearing polymerase and γ-phosphate-labeled nucleotides as described, for example, in U.S. Pat. Nos. 7,329,492 and 7,211,414 (each of which is incorporated by reference herein in its entirety), or nucleotide incorporations can be detected with zero-mode waveguides as described, for example, in U.S. Pat. No. 7,315,019 (which is incorporated by reference herein in its entirety), and using fluorescent nucleotide analogs and engineered polymerases as described, for example, in U.S. Pat. No. 7,405,281 and US Pub. No. 2008/0108082 (each of which is incorporated by reference herein in its entirety). The illumination can be restricted to a zeptoliter-scale volume around a surface-tethered polymerase such that incorporation of fluorescently labeled nucleotides can be observed with low background (see, e.g., Levene et al., Science, 299, 682-686, 2003; Lundquist et al., Opt. Lett., 33:1026-1028, 2008; and Korlach et al., Proc. Natl. Acad. Sci. USA, 105:1176-1181, 2008, each of which is incorporated by reference herein in its entirety). Images obtained from such methods can be stored, processed, and analyzed as set forth herein.

Some SBS examples include detection of a proton released upon incorporation of a nucleotide into an extension product. For example, sequencing based on detection of released protons can use an electrical detector and associated techniques that are commercially available from Ion Torrent (Guilford, Conn., a Life Technologies subsidiary) or sequencing methods and systems described in US Pub. Nos. 2009/0026082; 2009/0127589; 2010/0137143; and 2010/0282617, each of which is incorporated by reference herein in its entirety. Methods set forth herein for amplifying target nucleic acids using kinetic exclusion can be readily applied to substrates used for detecting protons. More specifically, methods set forth herein can be used to produce clonal populations of amplicons that are used to detect protons.

H. Data Analysis

Any appropriate bioinformatics workflow can be used to analyze and process the sequence reads obtained using the disclosed methods.

In some examples, reads originating from the same long DNA fragment are labeled with the same barcode to enable linked-read analysis. Since clusters pertaining to the same DNA fragment are spatially co-located on the flow cell, in relatively close proximity to the location where the bead was immobilized on the flow cell, accurate barcode assignment can be based on the identification of the bead locations (or “cluster patches”) on the flow cell. Bead location identification can be accomplished using real-time analysis (RTA) images directly (e.g., available on the Illumina MiSeq™ platform) and/or using the cluster coordinates reported by RTA. Thus, this workflow can be used, for example, on platforms supporting interrogation of cluster coordinates.

In some examples, given RTA (x,y) read coordinates on each tile (considering both surfaces and all tile swaths when appropriate), density-based spatial clustering can be performed to identify the bead locations on each tile, where each bead is assumed to correspond to a high-density cluster of reads compared to the lower-density background created by the reads that leaked into the interstitial space. The clustering procedure detects an unknown number of clusters (since the number of beads on each tile is not fixed), handles variable cluster shapes and sizes (in the event that the beads are not in a consistent size and a circular shape post-melting), and categorizes interstitial reads as noise. Any appropriate density-based clustering algorithms can be used to define clusters, for example the DBSCAN clustering algorithm. In several implementations, the bead boundary is computed from each resulting cluster by finding the convex hull of the points assigned to the cluster. To enhance the clustering results, a density-based read filtering procedure may be applied prior to clustering, which eliminates reads based on the sparsity of their neighborhood on the tile (for example, a read is filtered out if there are fewer than n other reads within a radius r around it on the tile, where n and r are configurable parameters). In some examples, a manual curation step to assess and correct the final results of the clustering procedure may be implemented.

In additional examples, bead location is determined from RTA coordinates using deep learning. For example, the U-Net convolutional neural network architecture for image segmentation or an appropriate convolutional neural network (CNN) model can be used to determine cluster patch boundaries and corresponding bead location. In some such examples, the training dataset includes manually annotated images obtained from coordinate-based plots, as well as synthetically generated images. Synthetic data augmentation is achieved by applying a set of transformations to the manually annotated images; the transformations include shape deformations, size, number, and placement variations, as well as, inter- and intra-bead density variation.

When sequencing DNA from a known reference genome, genome alignment information can be used to further refine bead identification, rescue interstitial reads, and improve the resulting barcode assignment. For example, beads assigned to the same cluster can be further separated by considering genome windows to which their reads mapped, along with their spatial proximity. Alternatively, inter-bead cross-talk can be quantified by counting reads that map to the same genome window in neighboring beads; bead pairs with significantly high cross-talk can then be merged to improve island contiguity and performance in several target applications, such as phasing. Probabilistic or “soft” barcode assignment is also considered for further performance improvement in several target applications, such as phasing and assembly.

In some examples, post-identification, each detected bead is associated with a unique barcode and the reads contained within the bead boundary are labeled with this barcode. As a result, reads that originate from the long DNA fragments initially immobilized the same bead are assigned to the same barcode and can be linked during subsequent analysis. In particular, for human genome phasing, barcoded reads can be linked into islands (corresponding to longer DNA fragments from which the reads originated) using the proximity in their genome alignment positions (e.g., reads in the same barcode can be linked if they map close by on the human genome), enabling the reconstruction of much larger phase blocks. In genome assembly, barcoding information can be used to disambiguate repeats and significantly increase the assembly contiguity, e.g. by first mapping reads to the partially assembled contigs and then using the barcoding information to link the contigs. Phasing and assembly pipelines have been implemented as subsequent steps of the data analysis workflow following the best practices for linked-read analysis.

### EXAMPLES

### Example 1. PCL Bead Preparation and Use

PLC beads of 3 μm average diameter can be purchased (for example from Phosphorex, Mass.). To functionalize PCL beads, active amino groups are introduced to the microsphere surface by aminolysis in a 10% (w/w) isopropanol solution in 1,6-hexanediamine at 40° C. for 60 min (as described in Yuan et al. J. Mater. Chem. B 3:8670-83 (2015)). Active amines on PCL bead surfaces are then conjugated to (i) amines on lysine residues of streptavidin and (ii) amine-functionalized magnetic nanoparticles by glutaraldehyde (FIG. 1A) (See Fang et al. RSC Adv 6:67875-82 (2016) and Hassan et al. Nano Res 11(1):1-41 (2018)). Biotin-conjugated transposomes are assembled on the surface of PCL beads by biotin-streptavidin binding (FIG. 1B). These biotin-conjugated transposomes may comprise polynucleotides conjugated to biotin. PCL beads are then ready for use in DNA fragmentation and library preparation and on-flow cell spatial cluster clouds generation.

PCL beads are introduced into the flow cell, where remaining streptavidin on PCL microsphere surface binds to biotin patterned on flow cell surface and immobilizes beads (FIG. 2). After release of the library and clustering, the PCL beads are released from the surface by excess free biotin. PCL beads are then melted at a temperature above 60° C. before removal from the flow cell by washing. Alternatively, alkaline hydrolysis with NaOH at high temperature can be used to degrade PCL beads (See Ramirez Hernandez et al. Am J Polym Sci, 3(4):70-75 (2013)).

### Example 2. Use of Compositions Comprising Beads and Nanoparticles

A mixture of compositions comprising a bead and at least one nanoparticle (wherein each nanoparticle comprises one or more transposome complexes) may be used to prepare a library for sequencing. Such compositions may be any of those described herein.

FIG. 8 provides a summary of a representative method of preparing a sequencing library using a mixture of compositions comprising a bead and at least one nanoparticle. A target nucleic acid (such as high molecular weight genomic DNA) is added to the composition and tagmentation is performed at 55° C. The tagmentation is stopped with 5% SDS solution. At this point, library fragments will be immobilized on nanoparticles. A magnetic force is applied to immobilize compositions (wherein the beads may be magnetic beads), and the supernatant is removed followed by 3 washes.

The reaction vessel is heated to 80° C. to release the clustering nanoparticle and the Tn5 transposase. Magnetic beads can then be separated from the rest of the reaction using a magnetic force. At this point, library fragments would be in solution and can be amplified in solution. If the compositions comprised identical transposomes, a step to incorporate a second adapter sequence may be performed before amplifying. The amplified fragments can then be loaded onto a flow cell for sequencing. Library fragments may be generated such that they have complementary adapter sequences to bind oligonucleotides immobilized on a flow cell.

Alternatively, compositions comprising beads may be loaded on a flow cell and then library fragments are released and captured on the flow cell. Such fragments may be generated with asymmetrical tagmentation that incorporates different adapter sequences using different transposome complexes. Beads may then be removed, such as by degrading with increased temperature or a degrading agent (if the composition comprises degradable beads as described herein). The immobilized fragments can be amplified in the flow cell, followed by sequencing. In all these methods, amplification before sequencing may also be omitted.

As shown in FIG. 8, a method with a mixture of compositions comprising a bead and at least one nanoparticle can avoid the cost and time that is normally spent on size selection of library fragments. In this method, the steric hindrance from multiple nanoparticles being comprised on a single bead allows for a method that avoids generation of short fragments by spacing the transposome complexes. Accordingly, sequencing may be performed with well-known methods for long read sequencing.

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5 or +/−10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

What is claimed is:

1. A degradable polyester bead comprising a plurality of transposome complexes immobilized to the surface thereof, wherein each transposome complex comprises a transposase bound to a first polynucleotide and a second polynucleotide,
wherein the first polynucleotide comprises a 3′ portion comprising a transposon end sequence and a tag, and the second polynucleotide comprises a 5′ portion that is complementary to and hybridized to the transposon end sequence, and
wherein the polyester bead has a melting point of from 50° C. to 65° C., optionally wherein the polyester bead has a melting point of 60° C., optionally wherein the polyester bead comprises polycaprolactone.

2. The degradable polyester bead of claim 1, comprising a plurality of magnetic nanoparticles immobilized thereto, optionally wherein the magnetic nanoparticles are beads with a magnetic core, optionally wherein the magnetic core comprises iron, nickel, and/or cobalt.

3. The polyester bead of claim 1, wherein each transposome complex comprises a polynucleotide binding moiety, the bead comprises a plurality of bead binding moieties covalently bound to the surface thereof, and the transposome complexes are immobilized to the bead surface through binding of the polynucleotide binding moieties to the bead binding moieties.

4. The polyester bead of claim 3, wherein:
a. each polynucleotide binding moiety is covalently bound to the first polynucleotide of each transposome complex or covalently bound to the second polynucleotide of each transposome complex;
b. the bead binding moiety is streptavidin or avidin and the polynucleotide binding moiety is biotin; and/or
c. each bead binding moiety is covalently bound to the polyester bead through a linker, wherein the linker optionally comprises —N═CH—(CH2)3-CH═N—, —C(O)NH—(CH2)6-N═, or —C(O)NH—(CH2)6-N═CH—(CH2)3 CH═N—.

5. The polyester bead of claim 2, wherein each magnetic nanoparticle is covalently bound to the polyester bead through a linker, wherein the linker optionally comprises —N═CH—(CH2)3-CH═N—, —C(O)NH—(CH2)6-N═, or —C(O)NH—(CH2)6-N═CH—(CH2)3 CH═N—.

6. The polyester bead of claim 1, wherein the polyester bead is immobilized on the surface of a flow cell, optionally wherein the polyester bead is immobilized on the surface of the flow cell through covalent binding of a bead binding moiety to a flow cell binding moiety on the surface of the flow cell.

7. The polyester bead of claim 6, wherein the polynucleotide binding moiety and the flow cell binding moiety are the same type of binding moiety, and the transposome complexes are bound to a first portion of the bead binding moieties on the bead and the flow cell binding moiety is bound to a second portion of the bead binding moieties on the bead.

8. The polyester bead of claim 1, comprising a target nucleic acid or one or more fragments thereof, each bound to at least two transposome complexes on the bead, optionally wherein most transposome complexes are immobilized on the surface of the bead.

9. A flow cell comprising a polyester bead immobilized to the surface of the flow cell, wherein the polyester bead comprises a plurality of transposome complexes immobilized to the surface thereof,
wherein each transposome complex comprises a transposase bound to a first polynucleotide and a second polynucleotide,
wherein the first polynucleotide comprises a 3′ portion comprising a transposon end sequence and a tag, and the second polynucleotide comprises a 5′ portion that is complementary to and hybridized to the transposon end sequence; and
wherein the polyester bead has a melting point of from 50° C. to 65° C., or 60° C., optionally wherein the polyester bead comprises polycaprolactone and/or comprises a plurality of immobilized magnetic nanoparticles immobilized thereto.

10. The flow cell of claim 9, wherein each transposome complex comprises a polynucleotide binding moiety, the bead comprises a plurality of bead binding moieties covalently bound to the surface thereof, and the transposome complexes are immobilized to the bead surface through binding of the polynucleotide binding moieties to the bead binding moieties and optionally wherein:
a. each polynucleotide binding moiety is covalently bound to the first polynucleotide of each transposome complex or covalently bound to the second polynucleotide of each transposome complex;
b. the bead binding moiety is streptavidin or avidin and the polynucleotide binding moiety is biotin;
c. each bead binding moiety is covalently bound to the polyester bead through a linker, wherein the linker optionally comprises —N═CH—(CH2)3-CH═N—, —C(O)NH—(CH2)6-N═, or —C(O)NH—(CH2)6-N═CH—(CH2)3 CH═N—;
d. the polyester bead comprises a plurality of immobilized magnetic nanoparticles immobilized thereto, and each magnetic nanoparticle is covalently bound to the polyester bead through a linker, wherein the linker optionally comprises —N═CH—(CH2)3-CH═N—, —C(O)NH—(CH2)6-N═, or —C(O)NH—(CH2)6-N═CH—(CH2)3CH═N—, and/or wherein the magnetic nanoparticles are used for seeding the polyester bead to a surface of the flow cell; and/or
e. the polyester bead is immobilized on the surface of the flow cell through covalent binding of a bead binding moiety to a flow cell binding moiety on the surface of the flow cell or wherein the polynucleotide binding moiety and the flow cell binding moiety are the same type of binding moiety, and the transposome complexes are bound to a first portion of the bead binding moieties on the bead and the flow cell binding moiety is bound to a second portion of the bead binding moieties on the bead.

11. The flow cell of claim 9, comprising a target nucleic acid or one or more fragments thereof, each bound to at least two transposome complexes on the bead, optionally wherein most transposome complexes are immobilized on the surface of the bead.

12. A composition comprising a bead and at least one nanoparticle, wherein the bead comprises a functional group that is capable of binding to the nanoparticle, optionally wherein the nanoparticle or the bead is magnetic.

13. The composition of claim 12, wherein the nanoparticle:
a. is a synthetic dendron, a DNA dendron, or a polymer brush; and/or
b. is a bead with a magnetic core, optionally wherein the magnetic core comprises iron, nickel, and/or cobalt; and/or
c. has a diameter of 50-150 nm, optionally wherein the nanoparticle has a diameter of 100 nm.

14. The composition of claim 1, wherein the nanoparticle comprises:
a. a single immobilized transposome complex, or
b. more than one immobilized transposome complex, optionally wherein the more than one immobilized transposome complexes are immobilized with similar distances between each transposome complex on the nanoparticle.

15. The composition of claim 14, wherein the immobilized transposome complex or transposome complexes are oriented with the transposase facing away from the nanoparticle.

16. The composition of claim 14, wherein the transposome complex is immobilized to the nanoparticle by:
a. binding of a transposon comprising biotin, desthiobiotin, or dual biotin to avidin or streptavidin comprised on the nanoparticle, or
b. a click chemistry reaction between an agent comprised in a transposon and an agent comprised in the nanoparticle, optionally wherein the click chemistry reaction is a reaction between an azide on the nanoparticle and a dibenzylcyclooctyne (DBCO) on the transposon.

17. The composition of claim 12, wherein the bead is a carrier bead that can bind multiple nanoparticles, optionally wherein the bead has a diameter of 1 μm or larger and/or the bead is a degradable polyester bead.

18. The composition of claim 12, wherein the functional group is a chemical attachment handle and/or a clustering primer, optionally wherein:
a. the chemical attachment handle and/or clustering primer directly binds to the nanoparticle;
b. the chemical attachment handle and/or clustering primer indirectly binds to the nanoparticle; or
c. a chemically modified oligonucleotide binds to both the clustering primer comprised in the bead and to the nanoparticle.

19. The composition of claim 12, wherein the interaction between the nanoparticle and the bead is a reversible and/or non-covalent interaction, optionally wherein the reversible and/or non-covalent interaction is a protein-ligand interaction or a metal-chelator interaction, further optionally wherein the protein-ligand interaction is a biotin-streptavidin interaction or the metal-chelator interaction is nickel-polyhistidine or cobalt-polyhistidine interaction.

20. The composition of claim 12, wherein the bead comprises a clustering primer and the nanoparticle comprises an immobilized oligonucleotide, optionally wherein the immobilized oligonucleotide and the clustering primer bind directly to each other or a linking oligonucleotide is capable of binding to both the immobilized oligonucleotide and the clustering primer.

21. The composition of claim 12, wherein the interaction between the nanoparticle and the bead is an irreversible and/or covalent interaction, optionally wherein the covalent interaction is a cleavable linker between the bead and the nanoparticle, further optionally wherein the cleavable linker is a chemically or enzymatically cleavable linker.

22. A flow cell comprising the composition of claim 12 immobilized to the surface of the flow cell, optionally wherein the composition is immobilized to the flow cell through binding of the nanoparticle to the surface of the flow cell.

23. The flow cell of claim 22, comprising a target nucleic acid or one or more fragments thereof, each bound to at least two transposome complexes immobilized on nanoparticles.
